(19) United States
(12) Patent Application Publication
(10) Pub. No.: US 2020/0383564 A1
(43) Pub. Date: Dec. 10, 2020

(54) METHODS BASED ON TEAR FILM BEHAVIOUR

(71) Applicant: BEYOND 700 PTY LTD, Castle Hill (AU)

(72) Inventors: Thomas James MILLAR, Roseville (AU); Burkhardt Siegfried SCHUETT, Petersham (AU)

(21) Appl. No.: 16/769,550

(22) PCT Filed: Dec. 8, 2017
(86) PCT No.: PCT/AU2017/000266

§ 371 Date: Jun. 3, 2020

(51) Int. Cl. A61B 3/10; A61B 3/14; A61B 3/00; A61B 5/00; A61B 3/12

(52) U.S. Cl. CPC A61B 3/101; A61B 3/14; A61B 3/12; A61B 5/4848; A61B 3/0016

(57) ABSTRACT

The present invention provides a method of diagnosing, or developing or monitoring a treatment regime for, an ocular condition in a subject based on detected physical behaviour in a tear film, or lack of tear film, in the subjects eye, the method comprising the steps of: (a) capturing from the subjects eye at least a first captured data set; (b) identifying at least a first comparative data set; (c) analysing the at least a first captured data set relative to the at least a first comparative data set, thereby detecting physical behaviour in the tear film; and (d) diagnosing, or developing or monitoring a treatment regime for, the ocular condition based on the detected physical behaviour of the tear film. Also provided are methods of selecting contact lenses, of evaluating the effects of wearing a contact lens, and of determining preferable wearing periods of contact lenses and rest periods from wearing contact lenses by subjects.

### TECHNICAL FIELD

The present invention relates to diagnosis and treatment methods for ocular conditions in a subject. More specifically, the present invention relates to diagnosis and treatment methods for ocular conditions, wherein the methods are based on tear film behaviour, and in particular on analysis of the physical behaviour of a tear film.

### BACKGROUND ART

Currently a widely accepted theory in the ophthalmic community is that the tear film covering the ocular surface of mammalian eyes is composed of three components: a mucin component; an aqueous component containing water, salts, proteins and nutrients; and a lipid component (Holly and Lemp 1977 Sury Ophthalmol 22:69-87; Chen et al 1997 Invest Ophthalmol Vis Sci 38:381-387) (3-layer model). According to the 3-layer model, these three components are, in the normal eye, typically stacked on top of one another and, together, form the tear film.

The total thickness of the tear film is about 3 μm (less than half the diameter of a single cell) with the lipid layer itself spanning the outer 70 nm. According to the 3-layer model, the aqueous component is effectively contained, being interposed between the mucin component and the lipid component, with the latter forming a blanket over the aqueous component, and inhibiting water within it from evaporating.

In Millar and Schuett ‘The real reason for having a Meibomian lipid layer covering the outer surface of the tear film-A review’, Experimental Eye Research, published online 14 May 2015, Vol. 137, pages 125-138, the authors undertook a literature review concerning the tear film, and research undertaken by leading groups in relation to the same.

The tear film re-forms each time after a blink. As the upper eyelid closes, the tear film fluid is pushed into a small space between the eyelid and the eyeball surface (lachrymal lake) from where it travels into the tear ducts that drain into the nose. The lipids covering the surface of the tear film are squeezed up. Upon eye opening, the upward movement of the upper eyelid spreads a new tear film onto the ocular surface with new tear film fluid coming from the lachrymal glands. The lipid layer is re-spread. Some new lipid is also added to the lipid film. This occurs because the glands which secrete the lipids are squeezed during the blink and express an amount of new lipids during the blink.

According to the 3-layer model, the tear film of a subject with dry eye disease or keratoconjunctivitis sicca cannot contain the aqueous component to the same extent as a tear film in a normal eye. Therefore, it is logical, according to this model, to diagnose the disease by determining how well the aqueous layer is contained (Pflugfelder et al 2000 Cornea 19:644-649; Jester (ed) 2004 Ocular Surface 2:53-168). One way of making the determination, sometimes called tear film break-up time or tear film stability, measures the time it takes for a tear film to break up after formation.

An important difficulty with this approach is that a determination about how long it takes for tear film break up is subjective and varies based on practitioner experience. Contributing to the difficulty is variability in diagnoses of normal and abnormal tear films. Surrogate measurements and arbitrary threshold/criterion are usually constructed in order to discriminate between an abnormal tear film and a normal one.

One typical surrogate measurement for tear film break-up time in the literature is evaporation rate. For example, several patents/patent applications are directed to diagnosing dry eye disease by estimating tear film break-up time/tear film stability from the supposed evaporation rate. That rate is calculated from measuring temperature changes in arbitrary manners and according to arbitrary formulas (US 2008/0174733, US 2012/0057126, US 2013/0079660).

Another recent article also bases its discrimination between normal eyes and dry eyes on temperature changes of regions in eyes, cooling rate of ocular surfaces, among other temperature statistics (Abreau, K. et al, ‘Temperatures of the Ocular Surface, Lid, and Periobital Regions of Sjogren's, Evaporative, and Aqueous-Deficient Dry Eyes Relative to Normals’, The Ocular Surface, January 2016, Vol. 14, No. 1, pages 64-73). The common thread between these prior art documents is that a diagnosis is sought to be made by estimating tear film break-up time/tear film stability based on temperature measurements and a statistical analysis of those measurements.

One currently recommended clinical practice for the diagnosis of dry eye is a sequence of steps as follows:
(a) an initial patient history;
(b) a general eye examination;
(c) a validated symptom questionnaire; and
(d) at least two objective tests to measure the status of the tear film, ocular surface, or Meibomian glands (Pflugfelder et al 2000 Cornea 19:644-649; Jester (ed) 2004 Ocular Surface 2:53-168; SWEENEY et al 2013 Exp Eye Res 117:28-38).

The questionnaire in step (c) relies on the patient's subjective view and scaling. Accordingly, the signs of dry eye are then measured by the objective tests in step (d).

Most of these tests are invasive and the tear film is adversely (artificially) affected by conducting the tests. Tear film break-up time involves putting fluorescein into the tear film. Measuring tear volume involves placing filter strips (Schirmer tests) or cotton threads (phenol red thread tests) in the eye. Measuring osmolarity (an indicator of excessive evaporation of the tears) involves collecting tears. To test for Meibomian gland dysfunction, the glands are manually expressed. Again, this does not indicate what naturally occurs in relation to Meibomian glands, nor does it give any real sense as to how the patient's Meibomian glands are functioning.

Moreover, the evaluation of the performance and stability of the tear film is indirect, costly and time consuming because it requires multiple diagnostic methods. It is vexing because it is difficult to correlate the results to symptoms described by the patients. Not only do these tests tend to have a relatively low predictive value of −70% (Wolffson JS at al. 2017. TFOS DEWS II Diagnostic Methodology report. The Ocular Surface 15: 539-574), they are also limited in their application to a discrete number of ocular diseases, most notably dry eye.

Currently, there is no single direct measurement or observation technique that can be used to evaluate the dynamic performance of the tear film so as to translate findings to clinical milestones in patient care. There is a need to provide less intrusive, more efficient and accurate methods of diagnosis in relation to ocular conditions, and of developing or monitoring a treatment regime for such conditions.

Contact lenses are a foreign material placed on the cornea. The cornea does not have a blood supply and so relies on the tear film to provide its oxygen. Accordingly, to ensure adequate comfort and preferred functioning, the tear film should, despite the presence of a contact lens, continue to function properly. The contact lens should behave as if it were the ocular surface and therefore, not interfere with the tear film.

In general, a class (and brand) of contact lens is chosen on the basis of the habits and desires of a customer. The class (and brand) is a matter of practitioner choice and, in practice, should be based on what s/he finds gives more comfort to the patient.

There are soft and hard contact lenses. Hard lenses are rarely used these days. The main classes of soft lenses are as follows:

Daily Wear (‘Dailies’): Discarded every night and replaced every morning. They are typically hydrogels, being plastic that is very water absorbent and very soft. They require much less care because they do need to be washed or stored overnight.

Weekly Wear (‘Weeklies’): Like daily disposable contact lenses, they are made to be worn for a week and then replaced with a fresh pair. They do not need to be washed or stored overnight.

Monthly Disposable (“Monthlies”): Very common and are removed every night, disinfected with a solution, and stored in a proper receptacle. They are then discarded and replaced at the end of a month.

Extended Wear: Worn continuously day and night for up to seven days and six nights, or one month. The seven day contact lenses are typically worn for six days and nights and then stored in a contact lens case for cleaning while the eyes are allowed to rest. The monthly contact lenses are typically made from a silicone hydrogel, which is generally tougher than the hydrogels used for the Dailies. They have high oxygen permeability. It is important to adhere to the wear schedule for each brand and type of extended wear contact lenses as it can differ from brand to brand.

Contact lenses absorb proteins, lipids and other components from the tears (become contaminated), as well as pollutants and allergens from the atmosphere, oils and soaps from the hands. Therefore, there is a need for them to be cleaned.

Selection of a contact lens typically involves an eye examination that includes the following steps. As will become apparent, the process of selection typically occurs over time culminating in the writing of a prescription:
(a) A history: General questions about the patient's lifestyle, the answers to which can guide preferences regarding contact lenses; and
(b) A comprehensive eye examination for visual acuity and eye health.
If the patient has poor hygiene, is likely to suffer from hay fever or other allergy, does not have (or comply with) routines, Dailies are typically recommended. For patients who have problems/difficulties inserting and removing contact lenses, then Extended Wear is likely to be the more preferred choice.
(c) Determining the fit:
   a. a keratometer is typically used to measure the curvature of the cornea. This is typically based on measuring a small region of the cornea;
      i. if the curvature of a contact lens is too flat or too steep for the patient's eye shape, it could cause discomfort or even damage to the eye;
   b. a topographer is used to provide extremely precise details about surface characteristics of the entire cornea.
      i. corneal topography measurements are sometimes combined with wavefront measurements which provide specific information about how well the eye focuses light. These combined measurements can help determine the type of contact lenses that will give the sharpest vision.
   c. the pupil and iris are measured to determine the best diameter of the contact lens. Preferably, a suitably fitting contact lens just covers the cornea.
(d) Tear film evaluation:
   a. tear production using Schirmer strips (although this is not a common test in optometry practices);
   b. tear break up time using either fluorescein or a slit lamp.
      i. if a severe dry eye condition is detected, the patient would be recommended to avoid or discontinue contact lens wear;
      ii. if a mild dry eye condition is detected, special contact lenses may be used.
(e) Testing a trial contact lens:
   a. A slit lamp is used to evaluate the fit of a trial contact lens to observe the alignment and movement of the lens as it rests on the surface of the eye;
   b. The examination is carried out several minutes after insertion of the trial lenses so that initial tearing of the eye stops and the lens stabilises.
(f) Testing comfort:
   a. This is generally done as an iterative process once the decision has been made about the main class of contact lens and shape;
   b. The patient will try several different brands for comparison and then choose a brand that offers them comfort. This requires follow-up visits;
   c. Comfort may be due to the shape of the contact lens edge (at its area of interaction with the ocular surface), breathability and wettability of the contact lens.
      i. the wettability is how readily the tear film interacts with the contact lens;
   d. Deposit build up on Extended Wear lenses can also be a reason for discomfort.
      i. These deposits will affect how the tear film interacts with the contact lens.
(g) Follow-up visits:
   a. Testing comfort:
      i. Fluorescein staining without the contact lens to see if the corneal surface has been damaged by the contact lens.
      ii. General questions about comfort
(h) Prescribing
   a. After finding a contact lens that fits properly, is comfortable, and provides good vision, a prescription is written.
      i. A prescription typically describes the contact lens power, a shape matching the curvature of the patient's eye (base curve), and a diameter for the lens (not a brand).

There are many brands of contact lenses, and each has unique features. There is no one brand that is best for all wearers.

Moreover:
(1) the interaction between the tear film and the contact lens is so important and can impact fit, comfort, visibility, reliability and/or general functionality;
(2) different patients have different needs in so far as contact lens make-up is concerned and, it is possible, that different contact lenses will suit different eyes in the same patient;
(3) there is a significant variety amongst different brands of contact lenses.

There is a need for improved methodologies for selecting suitable contact lenses for patients to wear.

Any reference to or discussion of any document, act or item of knowledge in this specification is included solely for the purpose of providing a context for the present invention. It is not suggested or represented that any of these matters or any combination thereof, formed at the priority date, part of the common general knowledge, or was known to be relevant to attempt to solve any problem with which this specification is concerned.

### SUMMARY OF INVENTION

The inventors have identified a novel method from analysing tear film behaviour and using the analytical results for clinical benefit. Interestingly, applying the novel methods, they have observed that tear film behaviour is not consistent with the 3-layer model, broadly due to the following findings:
1. a section of the tear film can be dragged around the corneal surface of the eye by using threads from the tip of a cotton bud which would be unlikely, if not essentially impossible, if the tear film has a distinct aqueous layer;
2. using the tip of an eyelash, the tear film could be disrupted in a single location. The disruption did not repair instantly or during a subsequent blink as it would if the tear film has a distinct aqueous layer.
3. by vigorous saline irrigation using a pipette or by using the edge of filter paper, a proportion of the tear film could be mechanically removed. It took from several blinks to up to about 1 hour for re-formation of the tear film over the corneal area under which that proportion of the tear film had been mechanically removed. This is inconsistent with the 3-layer model because in the 3-layer model the tear film's aqueous and lipid layers would be replaced during a blink and, as such, the portion of the tear film which had been removed, would be replaced.
4. a “sponge-squeezing”-like effect was caused by a hard blink. The tear film does not re-form normally after a hard blink compared to a normal unforced blink, because a hard blink causes more of the aqueous component of the tear film to appear. The expectation in a 3-layer model of the tear film would be that after a hard blink more lipids and aqueous would be released from the Meibomian glands and lachrymal gland respectively, which would improve tear film performance, including spreading.
5. adding artificial tear fluid (an isotonic buffered aqueous) resulted in the added artificial fluid being immediately removed via the puncta, while adding artificial tear fluid to the eye lid margin of an open eye, was not immediately removed, even after a blink. In a 3-layer model, added artificial tear fluid on the eyelid margin would be forced into the tear film during a blink leading to the artificial tear fluid integrating/comingling with, and being taken up by, the discrete aqueous layer. Any excess fluid in the tear film would immediately be removed via the puncta.
6. stimulated tearing resulted in excess aqueous visible after a blink as it evaporated. If the 3-layer model would be correct, the excess fluid which is not removed via the lachrymal duct would integrate into the aqueous of the tear film, covered by the tear film lipid layer and would not evaporate.
7. slow opening of the eye resulted in the tear film not forming properly because the lower sheer force applied by the upper eyelid during a slow blink, is not sufficient to cause the visco-elastic tear film to spread. In the 3-layer model after a slow blink both the aqueous and lipid layers could still spread across the ocular surface and form a normal tear film despite a slow rate of eye opening.

The inventors propose that the tear film is a gel shell like structure covering the surface of the eye.

Key constituents forming this gel shell like structure are mucins, which are found in relatively high concentrations in the eye. In general, mucins are highly glycosylated proteins with a propensity to bind water molecules and to interact with each other. Water containing mucin then meshes with other proteins and lipids forming integrated structures resembling a gel shell like structure, which the inventors, in some instances, call mucus.

In preferred embodiments, the mucus has a non-Newtonian behaviour and can be described as being viscous (for example, a measure of resistance to flow) and elastic (for example, a measure of stiffness). Counterintuitively, in preferred embodiments, the mucus changes one of its properties so as to become less viscous (for example, more fluid). The application of sheer forces preferably renders the mucus a lubricant that is spreadable.

In greater detail (with reference to an unforced blink in a normal eye), during eye closure in a blink, the mucus is preferably squeezed by the downward movement of the eye lid and a sheer force is preferably applied on the mucus structure. This is how, in some embodiments, the mucus changes one of its properties so as to become less viscous (for example, more fluid). In preferred embodiments, during this process, part of this mucus, preferably that part made of non-cell bound mucins with integrated water, is flushed out.

During eye opening, the components of the mucus which were removed in the downward blink are preferably replaced with secretions from goblet cells and secretions from other glands connected to the outside of the eye ball. Preferably, the upward blink applies a sheer force on the mucus, changing one of its properties so it becomes less viscous, and again preferably behaves like an aqueous fluid (preferably in lubricant form) that is spreadable. The tear film lipids preferably facilitate the spreading process during eye opening.

As the blink comes to an end, with the eye finally opening fully, the mucus preferably re-forms while parts of its components have been replaced. The gel shell like structure is again re-formed since a sheer force is no longer being applied.

In preferred embodiments, a difference for the gel shell model (relative to the 3-layer model) is that evaporation of tears is not prevented by the lipid layer covering the aqueous layer. Rather, it is the incorporation and integration of the aqueous into the mucus that holds the aqueous in the tear film.

The gel shell tear film model focuses on a different methodology to diagnosis of an ocular condition than the complex and subjective analysis undertaken based on the 3-layer model. As will become apparent from the content of this patent specification, the gel shell model also has utility in developing and monitoring a treatment regime for an ocular condition and for selection of suitable contact lenses for a patient.

According to a first aspect, the present invention provides a method of diagnosing, or developing or monitoring a treatment regime for, an ocular condition in a subject based on detected physical behaviour in a tear film, or lack of tear film, in the subject's eye, the method comprising the steps of:
a. capturing from the subject's eye at least a first captured data set;
b. analysing the at least a first captured data set and thereby detecting physical behaviour in the tear film; and
c. diagnosing, or developing or monitoring a treatment regime for, the ocular condition based on the detected physical behaviour of the tear film.

According to a second aspect, the present invention provides a method of diagnosing, or developing or monitoring a treatment regime for, an ocular condition in a subject based on detected physical behaviour in a tear film, or lack of tear film, in the subject's eye, the method comprising the steps of:
a. capturing from the subject's eye at least a first captured data set;
b. identifying at least a first comparative data set;
c. analysing the at least a first captured data set relative to the at least a first comparative data set, thereby detecting physical behaviour in the tear film; and
d. diagnosing, or developing or monitoring a treatment regime for, the ocular condition based on the detected physical behaviour of the tear film.

The detection of physical behaviour is, in preferred and alternative embodiments, achieved by visualising or observing captured data sets from a patient's eye. In some such embodiments, the visualisation or observation can be done on a screen, in recorded digital or analogue form, or in printed form, for example, in pictures and/or diagrams, all of these mechanisms being adopted with or without magnification means adapted to magnify the captured data set.

In some embodiments, the detection of physical behaviour occurs through capturing emissions and/or remissions within wavelengths from the electromagnetic radiation spectrum. In some preferred embodiments, detection occurs through infra-red emissions and/or remissions and visible light emissions and/or remissions.

In preferred and alternative embodiments, the detected physical behaviour is defined by one or more characteristics of the tear film, or lack of tear film, selected from the group consisting of: shape, size and position.

In some embodiments of the invention, the detected physical behaviour is defined by a shape or shapes of or in the tear film. The detected shape may be regular or irregular. It may additionally or alternatively fall within the range of well-defined to poorly defined. It may change from one form to another, or may continually change for a period of time, or be changing over time.

In some preferred embodiments, detected shape or shapes for a tear film is/are identifiable as associated with a particular status/es for an eye ranging from normal to having a condition. In some such preferred embodiments and in alternative embodiments, sub-ranges for one or more status/es may be identifiable in terms of detected shape/s, for example, such a sub-range may exist for an ocular disorder that is degenerating or one that has varying degrees of severity.

The tear film in a normal eye, by way of example only, immediately after an unforced blink is preferably detected to be substantially eye-shaped covering the air exposed surface of the open eye. This detected shape is preferably relatively stable for at least about three seconds after a blink, or more.

In some embodiments, the detected shape of the tear film in a normal eye, has an irregular portion detected on the medial side of the tear film between about 1 o'clock and about 6 o'clock positions for the patient's left eye and between about 6 o'clock and about 11 o'clock positions for the patient's right eye. In some such embodiments, that irregular portion is detected because the detection is done using thermal imaging technology, and the irregular portion represents a reflection of heat emanating from the side of the patient's nose. The irregular portion, in some preferred embodiments, is detected as moving toward the middle of the tear film increasing in size within about the first second after a blink. This could relate to the thermal imaging technology picking up an increasing amount of temperature from the patient's nose over time. Alternatively, it could relate to the presence of a disease or disorder.

In some alternative embodiments, the irregular portion is not detected as moving. In other embodiments, one or more irregularities are detected in the shape at different clock-face positions during the course of about the first second following a blink and for as long as about three seconds after the blink, or more.

By exemplary contrast only, the tear film in an eye affected by dry eye disease, immediately after a blink, may be detected to be similar in shape to that of the detected shape of the tear film in a normal eye but the detected shape is less well defined than is the case for the detected shape for a normal eye. Further, the detected shape of the tear film in mild to moderate dry eye becomes unstable (and/or becomes less well defined) faster than is the case with the detected shape of the tear film in a normal eye.

In some preferred and alternative embodiments, different outcomes in detection of shape are observed, in preferred and alternative embodiments, depending on the degree and aetiology of the dry eye condition. In some dry eye cases, for example, the detected shape of the tear film, or lack of tear film, is a substantially oval shape (cf eye shape). In some such embodiments, even over time, spanning as much as about 10 seconds or more, following a blink, the detected shape of the tear film is not changing, or only changing ever so slightly. In some such embodiments, the detected shape of the tear film is substantially consistent, even over a significant period of time, potentially exceeding about 10 seconds or more.

In some other examples, it is observed that in an eye with a corneal lesion, the detected shape of the tear film is irregular after around 1 second following a blink, and progressively becomes more regular toward the end of about 10 seconds after a blink, potentially due to the spreading of the tear film. In some such embodiments, the detected shape of a portion of the tear film adjacent the corneal lesion is shown tracking the outer limits of the corneal lesion.

In some further contrasting examples, it is observed that in an eye affected by Sjögren's syndrome, the detected shape of the bottom portion of the tear film may be regular along the edge of the ocular surface immediately after a blink and then becomes irregular after around 1 second after a blink.

Yet in some other examples or embodiments, it is observed that in a tear film of an eye with a shingles infection, the detected shape of the tear film is irregular adjacent the location of the infection immediately after a blink. Yet in some other embodiments or examples, it is observed that in an eye with keratoconus, the detected shape of the bottom portion of the tear film begins to show irregularity at around 0.4 seconds after a blink and this irregularity moves up and makes the shape of the whole tear film irregular at around 7.8 seconds after a blink.

In other examples, it is observed that administering eye drops to tear films may result in a previously irregular shape of a tear film changing to a regular shape.

Detected size of the tear film is another physical behaviour that is used alone or in combination with one of more of the other detected characteristics of tear film physical behaviour that is adopted by preferred and alternative embodiments of the present method. In some embodiments, the detected size can be relatively large, potentially taking up all or almost all of the corneal area exposed when a patient's eye is open. Toward the other end of the detected size spectrum, the detected size can be very small or non-existent, for example, in the case of a specific dry eye condition, where there may be an absence of tear film.

In some embodiments, the detected size of the tear film will change over time. By way of example only, eyes affected by dry eye disease will illustrate a detected tear film size that shrinks faster over time than that of the detected size of a tear film in a normal eye. In some other examples, it is observed that in an eye with a corneal lesion, the detected size of the tear film is considerably smaller immediately after a blink than would be the detected size of the tear film in normal eyes or in eyes with certain types of ocular disorders.

Yet in some other examples or embodiments, it is observed that in an eye after a hard blink, the detected size of the tear film may be considerably smaller than the detected size of the tear film after an unforced blink in the same eye. Yet in some other examples, it is observed that in an eye after around one week of eyelid blinking exercise, the detected size of the tear film remains stable for a longer period of time than the detected size in the same eye without (or prior to) having undertaken the around one week of eyelid blinking exercises. In yet other examples, it is observed that in an eye after treatment with an eye drop the detected size of a tear film becomes bigger than without that eye drop.

Preferred embodiments of the invention use detected position of the tear film in the eye alone or in combination with other detected physical behaviour/s, when employing the present method. Like, or in addition to, the other characteristics reflecting detected physical behaviour of a tear film, the detected position of the tear film can, in some embodiments, form a meaningful input to a diagnosis, or development or monitoring of a treatment regime in relation a particular eye condition. Preferred embodiments provide that the detected position may be relatively stationary or it may move over time, or during or after a period of time.

By way of example, in a normal eye, the detected position of a tear film is reflected by the tear film typically spreading across the corneal surface. In an eye of a subject who is a wearer of contact lenses, it is observed that the detected position of the tear film may disappear around the perimeter of the contact lens after around 0.5 seconds following a blink. In other wearers of contact lenses, the detected position of the tear film may only partially cover the inferior region of the contact lens. Yet in another example of an eye of a subject wearing a contact lens it can be observed that at the edge of the detected position of the contact lens the tear film may not form completely. Yet in another example of an eye of a subject wearing a contact lens the detected position of the tear film is affected by the movement of the contact lens due to eye ball movement. Here areas of the eye surface, which are normally not an exposed part of the tear film, can be affected.

In other embodiments or examples, it is observed that in an eye with a corneal lesion, the tear film detected position is away from the location of the corneal lesion. Yet in some other examples, it is observed that in an eye with a shingles infection, the tear film detected position is away from the location of the shingles infection. Yet in some other examples, it is observed that in an eye affected by Sjögren's syndrome, the tear film detected position is away from the bottom part of the eye during about the first second immediately after a blink.

By examining one or more of the detected shape, detected size and detected position of the tear film, preferably, at different times after a blink, the formation and stability of the tear film can be evaluated, according to preferred and alternative embodiments.

In some embodiments, for example, where the detected shape, detected size and detected position of the tear film covers the whole eye within less than a second and does not alter in appearance over many seconds, this may be regarded as a normal tear film.

In other embodiments, where after a blink the detected shape of the tear film is incomplete because, for example, it has not formed properly over individual regions or over multiple regions (for example, having a patchy appearance), or does not extend essentially all the way to the top of the eye (in this case, its detected shape and detected size are abnormal), it is considered to be a form of dry eye.

In still other embodiments, where the detected shape, detected size and detected position are abnormal, these detected characteristics may be indicative of a gross change to the ocular surface that could be caused by a known ocular condition such as, for example, keratoconus, a surface scar, or a contact lens on the ocular surface.

In further embodiments, where the detected position of the tear film changes with time, this may be indicative of insufficient tear film to maintain a stable tear film. An example of an ocular disorder in which this may occur is Sjôgren's syndrome where initially the detected shape, detected size and detected position of the tear film is normal, but then the detected position of the tear film preferably changes by gradually depleting from the inferior region of the eye.

In preferred and alternative embodiments, these various and different characteristics of tear film behaviour provide information about diagnosis and treatment of ocular conditions or of their absence. For example, when the detected shape, detected size and detected position of the tear film is such that it forms a normal tear film but does not extend to cover the superior region of the eye, that may be indicative of spreading of the tear film being incomplete. This symptom is commonly associated with insufficient lipids being incorporated in the tear film or an incomplete blink and can be treated accordingly.

In other examples and embodiments, where the detected tear film is patchy in appearance, that may be indicative of an underlying fault in the corneal epithelium, and possibly, their associated mucins and so the gel shell cannot form properly in these areas and can be treated accordingly.

In further examples and embodiments, where the detected shape, detected size and detected position of the tear film is affected by a contact lens, the method of the present invention provides that different contact lens brands can be trialled to determine a brand that is preferably suited to the wearer.

In some embodiments, detected tear film behaviour is conducted once off, continuously, and/or periodically. As is explained in more detail in this patent specification, in preferred and alternative embodiments, the capturing of the tear film behaviour is accomplished by observation, monitoring or recording. Preferably, practitioners can interrogate the observed detected tear film behaviour together with other comparative tear film behaviour to diagnose, develop or monitor a treatment regime for and ocular condition.

In some embodiments, the observing, monitoring, or recording of tear film physical behaviour are adopted by themselves of in combination.

In some embodiments, the capturing begins immediately after a blink because, in preferred embodiments, a substantial part of detected tear film physical behaviour occurs shortly after a blink, being, in some such embodiments, the time around which a tear film is formed. For example, the capturing of tear film physical behaviour may commence at around 0 second, immediately following a blink. In some other examples, the capturing may commence at a different time between 0 to 1 second following a blink, such as: 0.01, 0.02, 0.1, 0.2, 0.5, or 1 second or longer following a blink. Although it is preferred to begin the capturing immediately following a blink, in a tear film of an eye without an ocular condition, the tear film may remain stable for at least around a few seconds. In some embodiments, the capturing can begin after that amount of time. With that said, in some circumstances, the tear film may dissipate or become unstable more quickly than in other. Accordingly, in some embodiments, the capturing is commenced before, during, or as soon as possible following, a blink.

In some embodiments, depending on the type of ocular condition and/or the type of diagnosis, development or monitoring of eye conditions, the time period of capturing of relevant tear film physical behaviour may vary. The capturing may last for any period of time depending on the potential type of eye condition and the goal of diagnosis, developing or monitoring the eye condition.

For example, the capturing of tear film physical behaviour in an eye with dry eye condition, may last from 8 to 11 seconds following commencement of the capturing. In some other examples, the capturing of tear film physical behaviour in an eye of a subject wearing a contact lens, may last from 3 to 50 seconds following commencement of the capturing. Yet in some other examples, the capturing of tear film physical behaviour in an eye with Sjögren's syndrome, may last from 1.3 to 6 seconds following commencement of the capturing. Yet in some other examples, the capturing of tear film physical behaviour in an eye with a shingles infection, may last from 1 to 3 seconds following commencement of the capturing.

Yet in some other examples, the capturing of tear film physical behaviour in an eye having a corneal lesion, may last from 0.3 to 11 seconds following commencement of the capturing. Yet in some other examples, the capturing of tear film physical behaviour in an eye with keratoconus, may last from 0.4 to 8 seconds following commencement of the capturing. Yet in some other examples, the capturing of tear film physical behaviour in an eye before and after application of a specific eye drop, may last from 0.3 to 3 seconds following commencement of the capturing.

Yet in some other examples, the capturing of tear film physical behaviour in an eye before and after a hard blink, may last from 0.1 to 1 seconds following commencement of the capturing. In some other examples, the capturing of tear film physical behaviour in an eye before and after performing predetermined eyelid blinking exercises, may last from 1 to 7 seconds following commencement of the capturing.

Yet in some other examples, depending on the type of ocular condition and/or the type of diagnosis, development or monitoring of eye condition, the first or further capturing of tear film physical behaviour or lack of tear film, in a subject's eye may last a period of time, which period, following commencement of the step of capturing, is selected from the group consisting of: about 0.00 to about 1.00 second, about 0.00 to about 3.00 seconds, about 0.00 to about 6.00 seconds, about 0.00 to about 10.00 seconds, about 0.00 to about 15.00 seconds, about 0.00 to about 30.00 seconds, about 1.00 to about 7.00 seconds, about 3.00 to about 12.00 seconds, and about 6.00 to about 20.00 seconds. Yet in some other embodiments, the period during which tear film physical behaviour is captured lasts as long as the subject can maintain open the eye being examined.

In some embodiments, detected characteristics of tear film behaviour are captured in one segment or in multiple segments of the ocular surface. The capturing of multiple segments may commence at different times depending on the or each segment being captured, and the capturing may last for different periods of time depending on the or each segment being captured.

Preferably, all or a combination of the captured tear film physical behaviour characteristics are used to diagnose, develop or monitor a treatment regime for an ocular condition.

In some embodiments, depending on the type of ocular condition and/or the type of diagnosis, development or monitoring of eye condition, the second or further capturing may commence between 6 months and about one year following the preceding capturing. In some other embodiments, the second or further capturing may commence periodically following the preceding capturing, wherein the period may be a day, a week, a month, a quarter, a year, or as determined by a suitable practitioner.

Yet in some other embodiments, the timing and/or frequency for the second and/or further capturing is decided by the practitioner or the subject depending on various considerations such as: efficiency, diagnostic accuracy, response to or reaching a certain stage of treatment, for example, exercise regime, symptom/s, feeling/s, availability, response to certain types of medicines/eye drops, and response to certain types of ocular orthosis.

In some embodiments, an unforced blink is performed before or during the first or further capturing of tear film physical behaviour. In some other embodiments, in order to diagnose, develop or monitor eye condition, a variety of forces are applied to the blink before or during the first or further capturing of tear film physical behaviour. For example, a hard blink, or a blink using an intermediate level of force is performed during the first or further capturing of tear film physical behaviour.

In some embodiments, one or multiple set/s of comparative data is/are identified.

Preferred an alternative embodiments provide that the comparative data set/s is/are identified from one or more set/s of the captured tear film physical behaviour from the same subject, but at a different time or under a different condition or a combination of different time and condition, wherein the time or condition may be, among others: whether or not the subject is wearing a contact lens, the type/brand of contact lens the subject is wearing, the length of time for which the subject has been wearing a certain type of, or any, contact lens, the amount of force used in the blink during or before the capturing of tear film physical behaviour, whether or not eye drops are used before the capturing of tear film physical behaviour, the type of eye drop used before the capturing of tear film physical behaviour, the stage a treatment regime for an ocular condition has reached, or the progress of an exercise regime performed by the subject. Persons skilled in the art will appreciate that the timing and/or condition that is preferred to be adopted for when and how such comparative data set/s is/are captured can include a range of other timings and/or conditions.

In some embodiments, the one or more comparative data set/s is/are identified as being the result/s of having conducted one or more of the above or elsewhere explained capturing step of tear film physical behaviour in a different subject. For non-limiting example, the subject could be someone without any ocular condition, or someone with a certain type of ocular condition, or someone with a certain type of severe ocular condition, or someone, with or without an ocular condition, before or after a certain treatment/exercise regime. Again, persons skilled in the art will appreciate that the choice of different subject for the comparative data set/s can be made based on, for example, various theories or clinical observations.

In some other embodiments, the one or more comparative data set/s is/are identified from multiple different subjects, under different conditions, at different times.

In some other embodiments, the one or more comparative data set/s is/are identified from the other eye of the same subject whose eye is being analysed.

In some embodiments, the one or more comparative data set/s is/are identified as part of the knowledge base of one or more practitioners or people performing this invention. Preferably, that knowledge base includes training, studying, or experience of such person or persons. The knowledge base, used in some such embodiments, may be in the form of the memory of the person, or data in printed or digital form such as texts, tables, diagrams, pictures, images or videos in relation to tear film physical behaviour.

In some embodiments, the one or more comparative data set/s is/are identified by observing a predetermined source, wherein the predetermined source may be any material in relation to tear film physical behaviour such as: texts, photographs, video footage, medical or scientific imaging, and/or diagrams relating to detected/known physical behaviour in a tear film, or lack of tear film in a subject.

Preferred and alternative embodiments disclose that the identified comparative data set/s can be considered alone or in combination to assist the diagnosis, developing or monitoring eye condition. For a non-limiting example, a printed manual with texts, pictures, diagrams explaining and/or highlighting typical tear film physical behaviour in different conditions could be used to assist the person carrying out the invention. Video recording in the form of CD/DVD/Tape/computer file of tear film physical behaviour in different conditions could also be used as comparative data set/s.

In some preferred embodiments, the step of analysing includes evaluating the at least a first captured data set relative to the at least a first comparative data set to identify at least a first set of diagnostic characteristics. According to some such and alternative embodiments, taking into account that different eye conditions may have similar or overlapping diagnostic characteristics, a plurality of diagnostic characteristics may be used to differentiate between potential eye conditions, to settle a differential diagnosis, or to increase the likelihood that a diagnosis is accurate, for example.

In some preferred embodiments, wherein thermographic measurements contribute to the captured data set/s, as different components of a tear film have different emissivity, even at the same temperature, the detected physical characteristics of the tear film are preferably represented in grey scale. In some such embodiments that use a thermal sensitive camera to capture tear film physical behaviour, tear film configuration and its changes can then be visualised by the difference of grey scale in the capturing.

For example, in some preferred embodiments, a horizontal component of a tear film with lesser emissivity (darker) in a normal eye is detected to move from bottom to top between the opening of the eye and around 1 second after the opening of the eye. In some such embodiments, other configurations of the tear film components show little change, even after around 2/seconds or more after a blink. This preferably can be used to establish that aside from moving components, the tear film in a normal eye has a relatively stable configuration. In another example, it is observed in eyes with a dry eye condition that the moving component seen in the normal eye, moves much slower. That detected moving component does not reach the upper part of the ocular surface until after over around 5 seconds or more after a blink. In some such embodiments, the other configurations of the components of the eye are also detected as less stable. Changes can be seen after around 1 second or more after a blink.

In some embodiments, the analysing step is conducted by identifying potential correlations between the captured tear film physical behaviour and the comparative data set/s. One or more correlation may be identified by, for example, assessing the strength and extent of the correlation, or the similarity between the captured tear film physical behaviour and the comparative data set/s. In some embodiments, the preferred correlation/s is/are selected based on predetermined and/or preferred confidence intervals. In some other embodiments, identification of a relevant corresponding comparative data set/s may be performed by a person exercising his or her own judgment by observing such data set/s. In some other embodiments, computer programs may be deployed to facilitate or achieve analysis of the captured tear film physical behaviour and the comparative data set/s.

In some embodiments, diagnostic characteristics are identified for the captured tear film behaviour as the comparative data set/s have predetermined diagnostic characteristics associated with it/them. The diagnostic characteristics is/are, in some preferred and alternative embodiments, selected from the group consisting of: absence or presence of a possible ocular condition, differential diagnosis for a possible ocular condition, relative suitability of an ocular orthosis, relative efficacy of a treatment regime, and/or relative merit in maintaining, varying or ceasing a current treatment regime.

In preferred embodiments, one or more of the diagnostic characteristics are used as basis to diagnose, develop or monitor, an eye condition.

In some embodiments, the ocular condition is selected from the group consisting of: dry eye, aqueous deficient dry eye, evaporative dry eye, keratoconjunctivitis sicca, keratoconus, Meibomiam gland disorders, tear duct disorders, Sjôgren's syndrome, shingles or other ocular infection, corneal lesion, corneal scarring, Behcet's Disease, poor or incomplete blinking pattern, eye disease associated with rheumatoid arthritis, eye disease associated with connective tissue disorders, permanent or temporary closure of tear ducts and cosmetic variations. However, persons skilled in the art will appreciate that the invention is not limited to diagnosing or being used in the treatment of the above listed ocular conditions only.

In some other embodiments, with one or multiple diagnostic characteristic's identified in a subject's eye, if there is an existing treatment regime, the merit or effectiveness or such treatment regime can be evaluated. Different types/brands of ocular orthosis can also be evaluated base on their suitability to a subject. The diagnosis, monitoring, and evaluation can be one-off, at different times, or periodic depending on need or preference.

In some preferred embodiments, the ocular orthosis is an ocular device, a cosmetic variation or contact lens and developing or monitoring a treatment regime includes trialling different makes/models of such devices for the subject's eye or eyes.

In some embodiments, any one or more of the following steps can be conducted in real time: the capturing of tear film physical behaviour, identifying comparative data set/s, identifying a correlation between the captured tear film physical behaviour and the comparative data set/s, identifying the diagnostic characteristics and diagnosing, developing or monitoring ocular condition. In other embodiments, the method is conducted in real time.

In some embodiments, the capturing of the tear film behaviour is carried out by a video camera, for example, an infrared sensitive camera. Tear film physical behaviour is detected by the infrared sensitive camera partly due to the components or configuration of the tear film having different emissivities.

In some embodiments, the infrared sensitive camera used to capture the tear film physical behaviour is adapted to detect physical behaviour in wavelengths of from about 1.5 μm to 5.1 μm running at around 100 frames per second, with a spatial resolution of about 640×512 pixels, a pitch resolution of about 15 μm and a thermal sensitivity of about 15 mK.

In another example, the camera used to view the physical behaviour of the tear film is a Stirling engine cryocooled camera running at a frame rate of about 100 Hz using an indium antimonide sensor (approximately 640×512 pixel) with a pitch resolution of about 15 μm, a 1.5-5.1 μm spectral response, and with a 50 mm lens and 20 mm extension ring.

In another example, the camera used to view the physical behaviour of the tear film is an uncooled microbolometer running at a frame rate of about 60 Hz in a temperature window of about 20° C.−40° C. using a vanadium oxide sensor with about 320×240 pixels, a pitch resolution of 17 μm, a detector for wavelengths of from about 8 μm to about 14 μm, and a thermal sensitivity of about 20 mK, being equipped with an around 8.9 mm lens.

In other preferred embodiments, the camera may include a detector for wavelengths in a band range of between about 2 μm and about 14 μm.

In other examples, the camera has:
a. a frame rate of at least about 10 Hz, of at least about 25 Hz, or of at least about 60 Hz,
b. a spatial resolution of at about least 320×240 pixels,
c. a pitch resolution of about 17 μm or less, or of about 15 μm or less,
d. a thermal sensitivity of at least about 15 mK, of at least about 20 mK, of at least about 22 mK; of at least about 28 mK, or of at least about 35 mK.

Yet in other embodiments, the material of the lens system of the camera is selected from the group consisting of gallium, zinc selenide, or zinc sulfide.

Yet in other embodiments, the photodetector can be cooled and of different materials selected from the group consisting of: indium antimonide, indium arsenide, mercury cadmium telluride, lead sulfide, and lead selenide. In some embodiments, a Stirling engine cryocooler is used to cool the camera. However, gas coolers could also be used.

In preferred and alternative embodiments, the photodetectors include high band gap semiconductors, such as, for example, quantum well infrared photodetectors.

The digital information from the camera is, in some embodiments, processed by software. The software could be used to enhance the captured tear film physical behaviour. For non-limiting example, software computerising a mean of multiple frames into one frame could be used to increase the temperature sensitivity (for example, to reduce the noise); compare neighbouring pixels and perform statistical analysis for contrast enhancement or other enhancements. This software can be installed in a computer, in a camera or in a standalone device.

In some embodiments, a typical eye examination session proceeds as follows: The camera system is started and if necessary the camera is cooled to operational requirements. A computer with relevant software is started.

The patient is seated in front of the camera and asked to place his/her chin in a chin rest. The chin rest is adjusted for the patient to sit comfortably. The camera is adjusted to place it horizontally in front of the eye of the patient.

The camera can either work with a fixed focus or an adjustable focus. In the case of a fixed focus, the camera is moved on the horizontal axis to or away from the eye of the patient to get the thermographic picture in focus. If the focus of the camera is not fixed, additional adjustments can be made using the focal lens of the camera.

After the eye is in focus of the camera, the operator of the tear film thermographer gives instructions to the patient for blinking regimes and physical behaviour of tear film is captured and recorded as desired. This process can be repeated multiple times if needed or preferred.

The captured tear film behaviour is then analysed against a comparative tear film behaviour recorded in a database or a printed manual. A diagnosis is then made, or a treatment regime is developed or monitored, for the subject.

The abovementioned camera system can be fully motorized, manual, semi-autonomous or autonomous in operation. Such a tear film thermographer could be a stand-alone system or attached to another ophthalmic instrument allowing for movement of the camera into the right position in front of the eye of a patient. Such a system could be a slit lamp to which an infrared sensitive camera is attached so it can be moved as required.

According to a third aspect of the present invention, there is provided a method of selecting a contact lens for a subject, the method comprising:
a. capturing from a first eye of the subject a first captured data set, the first captured data set including detected physical behaviour of the tear film in the first eye;
b. identifying a first test contact lens and having the first test contact lens instilled in the first eye;
c. after a predetermined or preferred first period of time, capturing from the first eye a second captured data set, the second captured data set including detected physical behaviour of the tear film of the first eye with the first test contact lens instilled;
d. analysing the second captured data set relative to the first captured data set and/or a comparative data set; and
e. evaluating the relative suitability of the first test contact lens to be selected as the contact lens for the subject.

In preferred and alternative embodiments, the method of the third aspect further includes, after a predetermined or preferred second period of time, capturing from the first eye a third captured data set, the third captured data set including detected physical behaviour of the tear film of the first eye after the first test contact lens has been removed following an instilled period.

Preferably, the predetermined or preferred first period of time commences immediately following instillation of the first test contact lens and ends following initial tearing has subsided. In some embodiments, the predetermined or preferred first period of time is at least about 3 to about 5 minutes, and in other embodiments, the predetermined or preferred first period of time is selected from about 30 minutes, 1 hour, 2 hours, 4 hours, 6 hours, 8 hours, 24 hours, or longer.

According to further preferred and alternative embodiments, the method of the third aspect is conducted for the second eye of the subject using a corresponding first test contact lens and/or a different test contact lens.

Preferably, the method is repeated for a second test contact lens, and/or repeated for one or more further test contact lens.

In some embodiments, the method is repeated for a corresponding second test contact lens or further different contact lens.

In preferred embodiments, the method is performed on both of the subject's eyes simultaneously or substantially simultaneously.

In some preferred embodiments, a contact lens suitable for selection is one the removal of which allows the detected physical behaviour of a tear film to show detected physical behaviour substantially consistent with immediate re-establishment of a normal tear film.

In some preferred embodiments, a contact lens suitable for selection is one the removal of which allows the detected physical behaviour of the tear film to be close to undisrupted or only mildly disrupted, with re-establishment of normalcy in a relatively short period of time. In some such embodiments, the relatively short period of time is less than about 3 minutes.

In preferred and alternative embodiments, the installed time is from about 10 minutes to about 1 hour or longer.

In some preferred embodiments, a contact lens suitable for selection is one which results in a detected physical behaviour of a normal tear film initially on instillation of the contact lens and then continuing over time or one which does not result in a change in the detected physical behaviour of the tear film from before the fitting the contact lens.

In some embodiments, a contact lens less preferred for selection is one which does not allow the detected physical behaviour of the tear film to show normal formation of a normal tear film initially following instillation, but allows the detected physical behaviour of the tear film to show either a complete or partial tear film formation over time.

In further preferred and alternative embodiments, a contact lens preferred to be excluded from selection is one which results in the detected physical behaviour of a tear film appearing normal initially and then deteriorating over time.

In yet further preferred and alternative embodiments, a contact lens preferred to be excluded from selection is one for which the detected physical behaviour of tear film appears disrupted initially and remains disrupted over time.

Preferably, the method of the third aspect is repeated for a plurality of test contact lenses so as to select a contact lens for a subject. In some embodiments, the method is repeated for a plurality of corresponding test contact lenses and/or for a plurality of further different contact lenses so as to select a corresponding contact lens and/or a further different contact lens for a subject.

According to a fourth aspect, there is provided a contact lens selected according to the method of the third aspect.

### BRIEF DESCRIPTION OF THE DRAWINGS

Preferred embodiments of the invention will now be described and illustrated by reference to the accompanying drawings in which each figure shows a series of thermographs from a thermographic film, wherein the numbers in each frame represent time periods of 0.01 seconds:

FIG. 1 shows the moving tear film of a normal subject's eye following a blink.

FIG. 2 shows the tear film of a normal subject's eye moving along cotton bud fibres on the open subject's eye.

FIG. 3 shows the tear film of a normal subject's eye after an eyelash has been dragged through the tear film while the eye is open and, after that procedure, the subsequent blink.

FIG. 4 shows the appearance of the tear film of a normal subject's eye after washing the eye with an isotonic buffer compared to the tear film of the same subject's eye before the washing procedure.

FIG. 5 shows the tear film of a normal subject's eye following a blink after a filter paper has been dabbed on the surface of the eye while the eye was open and, after this procedure, the subsequent blink.

FIG. 6 shows the tear film of a normal subject's eye after the subject has carried out a hard blink.

FIG. 7 shows the tear film of a normal subject's eye after 2 μL of an isotonic artificial tear buffer was applied onto the eyelid margin while the subject's eye was open. Thermographic stills were taken directly after instilling the buffer, before the subsequent blink, and then during and after the subsequent blink.

FIG. 8 shows the tear film of a normal subject's eye after tearing was stimulated while the eye was open and the subsequent blink.

FIG. 9 shows the tear film of a normal subject's eye during and after a slow blink.

FIG. 10 shows a subject's eye, who had been diagnosed with dry eye, with incomplete spreading of the tear film following a blink.

FIG. 11 shows a subject's eye, who had been diagnosed with dry eye, with complete spreading, but unstable tear film, following a blink.

FIG. 12 shows a subject's eye, who had been diagnosed with dry eye, with no visible spreading of a tear following a blink.

FIG. 13 shows a subject's eye with Sjögren's syndrome after a blink.

FIG. 14 shows a subject's eye after a shingles infection.

FIG. 15 shows a subject's eye having a corneal lesion.

FIG. 16 shows a subject's eye, who had been diagnosed with keratoconus.

FIG. 17 shows a subject's eye before and after application of a specific eye drop.

FIG. 18 shows a subject's eye before and after application of another specific eye drop.

FIG. 19 shows a subject's eye, who had been diagnosed with dry eye, before and after performing blinking exercises for a week.

FIG. 20 shows a subject's eye after a blink, before and shortly after, instilling an extended wear contact lens.

FIG. 21 shows a subject's eye after a blink, before and shortly after, instilling a daily contact lens.

FIG. 22 shows a subject's right and left eyes after wearing a disposable daily contact lens for 6 hours in each eye.

FIG. 23 shows a comparison of the effects of different contact lenses on two different subjects after instilling the contact lens and during wear of the contact lens.

FIG. 24 shows a subject's right eye before, directly after, and during, contact lens wear and after removal of the contact lens.

### DETAILED DESCRIPTION OF EMBODIMENTS

It will be apparent that, in some preferred and alternative embodiments, several elements come together in the development of the present method. This observation is made not to limit in any way the subject-matter or scope of the invention, but rather to provide a framework for this detailed description. Broadly speaking, the elements that have come together are:
A) elements illustrating the gel shell model,
B) elements illustrating the use of the method in diagnosis or development or monitoring of a treatment regime, for ocular conditions, and
C) elements illustrating the use of the method in contact lens selection.

In the text that follows, a detailed description of embodiments of the invention will be provided by reference to these three elements.

A. Gel Shell Model Theory

The inventors theorise that the 3-layer model has a fundamental shortcoming because it cannot explain the ability of the tear film to resist evaporation. A common belief is that, in the 3-layer model, the tear film lipid layer on the air interface acts as a protective blanket to help the tear film to resist evaporation. This is not the case and of contention in the scientific community (Willcox MDP et al. 2017. TFOS DEWS II Tear Film Report. The Ocular Surface 15, 366-403), thus another mechanism must exist to assist the tear film resisting evaporation.

In seeking to substantiate their view regarding the 3-layer model and test their hypothesis of the new gel shell model, the inventors carried out a series of experiments. In these experiments, which will be referenced as Examples in the text that follows, subjects with an otherwise normal eye and normal tear film were asked to sustain an open eye after a blink. This was to ensure that a stable tear film could be formed and sustained stably for at least ten seconds to enable its behaviour to be observed. The tear film behaviour was recorded using a thermographic camera with an 640×512 indium antimonide detector array, a pixel pitch of 15 μm, a temperature resolution of 20 mK, a 50 mm lens with a 20 mm extension ring run with windowing at 100 Hz. The experiments were carried out in a controlled environment with a temperature of 23° C. and humidity of 45%. The thermographs are grey scaled, where darker grey represents less thermal radiation.

### Example 1: Normal Tear Film

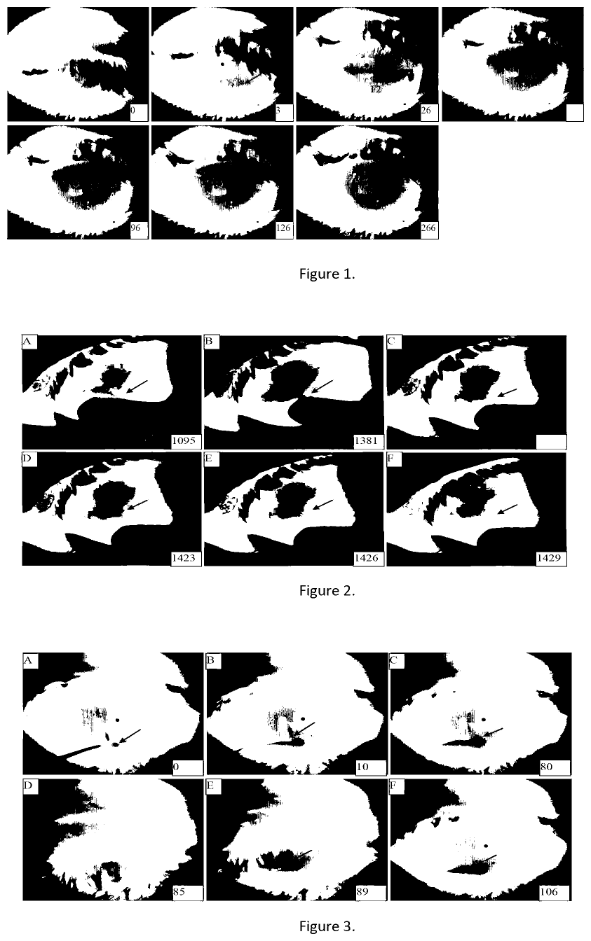

In one experiment, a subject with normal eyes was asked to sustain an eye opening for a period of time after an unforced blink. By using an apparatus suitable to carry out the present method, a thermographic video of the left eye of the subject was captured during this period. After a normal blink, the tear film can be observed to spread upwards from the bottom lid. This spreading is represented by the dark horizontal line (FIG. 1; F1) moving upwards following a blink. A light grey sheet (F2) follows behind this dark line. The horizontal line moves relatively rapidly and completely up the ocular surface, and afterwards there is virtually no change in the surface for many seconds if the eye is kept open. In some examples this was for more than 100 seconds.

### Example 2: Cotton Bud Swipe

A cotton bud was gently pressed against the edge of the ocular surface of a subject's eye so that of few fibres of the cotton bud were in contact with the tear film (FIG. 2, arrow). As the cotton bud was moved slowly across the ocular surface to one side of the eye, the inventors found that the whole tear film was dragged to the side of the movement of the cotton bud and its fibres in contact with the tear film. As the fibres from the cotton bud disengaged from the surface, the tear film relaxed back to its previous position

The finding that the entire tear film moved with the cotton bud is inconsistent with the 3-layer model as a tear film consistent with it, having a distinct aqueous layer, would be unlikely to, if not definitely would not, remain intact and be moveable as such in those circumstances. It could also be expected that if a 3-layer model were correct then aqueous would be absorbed by the cotton fibres and as such would change the tear film locally and would not be able to relax back to its former appearance. Alternatively, the lipid layer could be absorbed by the cotton fibres, exposing the aqueous layer below to evaporation. However, no darkening in the thermographic film was detected in the experiment.

By contrast, the finding that the entire tear film moved with the cotton bud supports the gel shell model because the mucus is a non-Newtonian fluid that is more elastic (for example, has a higher modulus of elasticity) than water. This higher elasticity of the mucus means that cotton fibres are able to distort the gel shell when gently dragged across the eye, and the gel relaxes back into its former state as soon as the force of the cotton bud is eased.

### Example 3: Eye Lash Swipe

The tip of an eyelash was gently struck across the surface of the subject's eye (FIG. 3). Footage captured during the swipe revealed that the eyelash disrupted the surface of the tear film in a distinct and single location (arrow). This disruption of the tear film does not cure and remains, even after a blink.

The finding that the disruption took time to cure is inconsistent with the 3-layer model. The disruption seen with thermography is likely due to evaporative cooling of aqueous. According to the 3-layer model both the aqueous and the lipid layer would instantaneously fill in behind the eye lash as it moved through the tear film. Even if the tear film lipid layer that sits on top of an aqueous layer would have been removed by the eye lash swipe, it would quickly spread into this disruption either spontaneously or during a blink to again cover the aqueous. If the lipid layer blanket theory would be correct, the evaporative cooling would disappear in either of those two situations.

By contrast a wound in a gel will not repair and the disruption of the mucus allows release of aqueous into that damaged area, which is then free to evaporate. A number of blinks would be required to cure the defect due to the viscoelasticity of the mucus while the eye is open and while the mucus is spreading.

### Example 4: Mechanical Removal of a Portion of the Tear Film

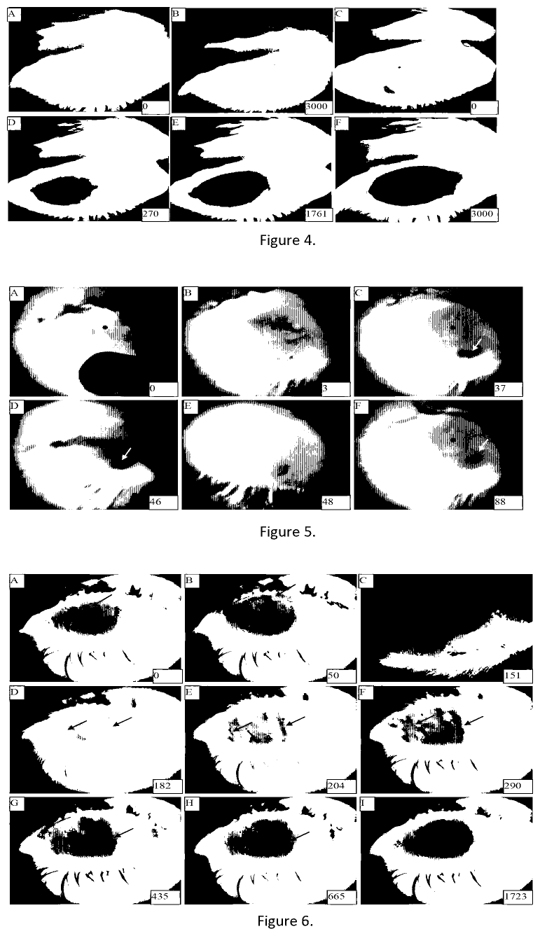

Using a pipette and local washing with isotonic saline (FIG. 4) or dabbing the edge of filter paper onto the ocular surface (FIG. 5), a proportion of the tear film from a subject's eye was removed. The tear film did not reform normally over the areas affected by these procedures. If the filter paper were dabbed onto the surface of the eye, it took several blinks for the defects in the tear film (arrow) to disappear. In the case of removing the tear film by washing with an isotonic saline solution, the tear film needed several minutes of normal blinking to appear normal again.

The finding that it took more than a blink or longer for the tear film to appear normal again is inconsistent with the 3-layer model because the fluidity of the layers should have instantly repaired the tear film in particular after a blink.

By contrast, the finding that the disturbance of the tear film is not repaired immediately and the reformation of the tear film takes time and several blinks is consistent with the tear film being a gel that needs time to reform once it is damaged or major parts of it have been removed from the eye.

### Example 5: Impact of a Hard Blink on the Tear Film

A subject with a normal tear film was asked to perform a hard blink. By using an apparatus suitable to carry out the present method, a thermographic video of the left eye of the subject was captured during this period. The effect on the tear film as the subject's eye opened and a period thereafter (FIG. 6). The tear film did not appear normal as areas of lower emissivity can be observed directly after the blink (arrows). These areas are not visible in the normal tear film (FIG. 1), but obvious after a hard blink for several seconds. Initially they get darker after the blink indicating that excess fluid is evaporating from the surface and then they get smaller and finally disappear. The vision of the subject after a hard blink is blurry compared to a normal blink but no difference in comfort was noticed by the subject holding the eye open for an extended time, though the tear film did not appear to be normal initially, indicating that the detected lower emissivity is not a result of aqueous content in the tear film evaporating.

The finding that the tear film did not appear normal following a hard blink compared to a normal unforced blink is inconsistent with the 3-layer model because the expectation would be that after a hard blink more lipids and aqueous would be released from the Meibomian glands and lachrymal gland respectively which should improve the tear film performance and the spreading of the tear film. In the 3-layer model the release of excess lipid should suppress evaporation, because the lipid layer would be thicker. The 3-layer model cannot explain evaporation happening immediately after a blink which over time subsides.

By contrast, the finding that the tear film appears not normal and the vision is blurry while the subject feels comfortable keeping the eye open following a hard blink, supports the gel shell model. In this model, the tear film gel is formed normally after the hard blink but excess of aqueous which is released after the hard blink is not incorporated in the gel shell. This excess fluid is not incorporated into the mucus, hence it evaporates creating areas with detected lower emissivity. Since the underlying tear film (the mucus) is intact, there is no perception of a faulty tear film (and, therefore, no discomfort) besides a blurry vision by the subject. Again here, if the lipid layer blanket theory would be correct, the excess lipids produced by the hard blink would cover the excess aqueous and evaporation would not occur.

### Example 6: Impact of Adding Artificial Tear Fluid on Tear Film

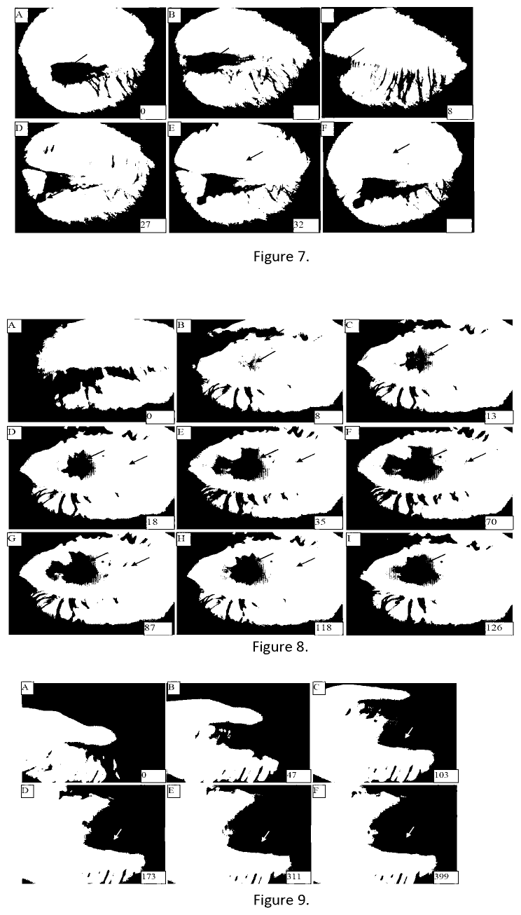

Artificial tear fluid was added onto the eye lid margin of an open eye (FIG. 7) immediately appearing as an area of lower emissivity on the eye surface after a blink (arrows) which indicates evaporative cooling. During the blink process, some of the artificial tear fluid is forced onto the skin outside the eye (arrow).

The finding that the added artificial tear fluid was immediately removed by the lachrymal lake is inconsistent with the 3-layer model. It would be expected that the added artificial fluid would integrate/comingle with, and be taken up by, the discrete aqueous layer. In particular, as a result of a blink, excess artificial tear fluid on the eyelid margin would be forced into the tear film and be expected to integrate/comingle with, and be taken up by the discrete aqueous layer.

By contrast, the finding that the added artificial tear fluid was immediately removed by the lachrymal lake supports the gel shell model because the amount of fluid in the gel is limited and because the gel already has an adequate amount of fluid. The artificial tear fluid added is not incorporated but instead moves to the lachrymal lake and this excess fluid is removed by the lachrymal ducts during the blink. The observation of excess artificial tear fluid on the lid margin being spread onto a local part of the ocular surface as a result of a blink forcing this excess fluid onto the ocular surface is consistent with the excess fluid being spread over the top of an existing gel shell and evaporating due to not being incorporated into the gel shell.

### Example 7: Impact of Excess Tearing on Tear Film

In a subject with a normal tear film additional tearing was stimulated while blinking was recorded. The tear film during and after a normal blink directly seen after the tearing has started did not appear normal and areas of lower emissivity were observed (FIG. 8). These areas (arrows) represent evaporative cooling of the excess aqueous from the eye while at the same time the subject did not notice any difference in comfort when holding the eye open compared to normal, although the normal tear film does not have these areas (FIG. 1).

The finding that excess tearing is visible as areas of enhanced evaporative cooling is inconsistent with a 3-layer model of a tear film. The excess tear would be incorporated in the aqueous of the tear film in this model and covered by the tear film lipid layer. In fact, stimulated tearing is discussed and tested as a possible treatment for dry eye.

By contrast, the finding that additional aqueous from stimulated tearing was creating areas of lower emissivity supports the gel shell model because the amount of fluid in the gel is limited and because the gel already has an adequate amount of fluid. The additional tear aqueous is not incorporated but part of it is removed through the lachrymal ducts and other parts of it are spread onto the ocular surface over the top of an existing gel shell, evaporating due to not being incorporated into the gel shell.

### Example 8: Impact of Slow Opening of the Eye Following a Blink

A subject with a normal tear film was asked to perform a blink such that the eye opening phase was very slow and footage captured the effect on the tear film as the subject's eye opened and for a period thereafter (FIG. 9). The tear film did not appear normal as areas of lower emissivity (dark lines) can be observed directly after the blink (arrows). These areas are clearly detected and get larger and expand (arrows). This is not seen in a healthy eye when the eyes are opened at a normal rate during a blink (FIG. 1). The subject feels discomfort and pain if the eye is kept open for an extended period of time.

This does not conform with the 3-layer tear film model as both the aqueous and lipids, both being fluid, should spread across the ocular surface and form a normal tear film despite a slow rate of eye opening.

By contrast, the gel shell has a non-Newtonian behaviour and so is less viscous when subjected to a high sheer force (by, for example, a fast blink) and more viscous when subjected to a low sheer force (by, for example, a slow blink). Therefore, during a slow eye opening after a blink, the mucus is more viscous and does not spread to cover the surface of the eye. In areas where it has not spread correctly, free fluid evaporates causing evaporative cooling. This evaporation from the ocular surface leads to drying of the ocular surface during extended eye opening, resulting in the subject feeling discomfort.

Taken together, these examples show that the tear film cannot be understood as a 3-layer entity with a distinct aqueous layer covered by a lipid layer, while the lipid layer is responsible for retaining the aqueous. According to this model tear film problems are related to the aqueous component evaporating at a higher rate compared to a normal tear film. Tear film break-up, and this evaporation is the means to monitor tear film stability.

With the gel shell model, mucins form a gel-like structure while binding aqueous. Similar to mucus found in other parts of the human body, the evaporation rate would be dependent on the quality of this mucus to retain the aqueous bound to it. This mucus is spread with the help of the lipids during a blink, which in turn suggests that a faulty spreading of the mucus would result in a faulty tear film. Thus, the observation of a faulty tear film could be achieved by detecting the physical behaviour of the tear film during the spread of the mucus in the course of a blink.

Also, in preferred and alternative embodiments of the gel shell model the mucus component of the tear film slows and/or inhibits evaporative loss of aqueous. By doing this, the mucus component of such embodiments, may stabilise the tear film. In some embodiments, additional and higher evaporative loss may come from aqueous not being properly incorporated in the mucus, such that the evaporation rate will, in some cases, depend upon the osmolarity of the aqueous that has not been incorporated into the mucus. The additional evaporation can, in some embodiments, be attributed to:
a. mucus of the tear film not being formed properly on parts of the surface of the eye exposed to air;
b. mucus not being spread properly over the entirety of the surface of the eye exposed to air; and/or
c. there being excess aqueous that is not incorporated into, or bound to, the mucus.

In some embodiments, after stimulated tearing, after a hard blink, or after addition of artificial tear fluid, a detected change in size, a detected change in shape, and a detected change in position may be attributed to evaporative cooling of unincorporated and/or unbound aqueous. In some such embodiments, evaporative cooling of the unincorporated and/or unbound aqueous can be detected and has preferably no or only minimal detrimental effect to the comfort of the subject and preferably no or only minimal effect on the stability of the underlying gel shell tear film. According to some such embodiments, those situations do not necessarily reflect an abnormal tear film. They can also occur in any of a number of conditions where an excess of aqueous is present in the eye.

B. Use of the Method in Diagnosis of, or Development or Monitoring of a Treatment Regime for, Ocular Conditions

In some preferred and alternative embodiments, there is provided a method of diagnosing, or developing or monitoring a treatment regime for, an ocular condition in a subject based on detected physical behaviour in a tear film, or lack of tear film, in the subject's eye, the method comprising the steps of:
a. capturing from the subject's eye at least a first captured data set;
b. analysing the at least a first captured data set and thereby detecting physical behaviour in the tear film; and
c. diagnosing, or developing or monitoring a treatment regime for, the ocular condition based on the detected physical behaviour of the tear film

In other preferred and alternative embodiments, there is provided a method of diagnosing, or developing or monitoring a treatment regime for, an ocular condition in a subject based on detected physical behaviour in a tear film, or lack of tear film, in the subject's eye, the method comprising the steps of:
a. capturing from the subject's eye at least a first captured data set;
b. identifying at least a first comparative data set;
c. analysing the at least a first captured data set relative to the at least a first comparative data set, thereby detecting physical behaviour in the tear film; and
d. diagnosing, or developing or monitoring a treatment regime for, the ocular condition based on the detected physical behaviour of the tear film.

Several experiments were conducted by the inventors with a view to illustrating the way in which the method of the invention can be used in a method in diagnosis of, or development or monitoring of a treatment regime for, ocular conditions. These experiments will now be described in some detail sequentially, referenced as examples. The text that follows the examples below will provide an explanation of the figures that were capture as part of the experimentation.

As overarching statements, some experiments were conducted on patients with different circumstances, such as:
a. patients with a particular ocular condition,
b. patients having undertaken a particular blinking exercise regime,
c. patients who had a specific eye drop administered to their eyes.

In each case, the patient was asked to sustain an eye opening after an unforced or hard blink. This was to allow sufficient time for detected physical behaviours of the tear film to be analysed and recorded. A thermographic camera with an 640×512 indium antimonide detector array, a pixel pitch of 15 μm, a temperature resolution of 20 mK, a 50 mm lens with a 20 mm extension ring run with windowing at 100 Hz. The experiments were carried out in different air-conditioned locations with slight variations in ambient temperature and humidity.

### Example 9: Dry Eye Case Study 1

A subject with a dry eye condition was asked to sustain an eye opening for a period of time after an unforced blink. A thermographic video of the right eye of the subject was captured. As illustrated in FIG. 10, a detected tear film was formed immediately after the eye opening, but the detected shape, detected size, and detected position of the tear film began to change at around 0.2 seconds and became obvious at 0.7 seconds after the eye opening, showing a less stable detected physical behaviour of the tear film than that of a normal eye.

### Example 10: Dry Eye Case Study 2

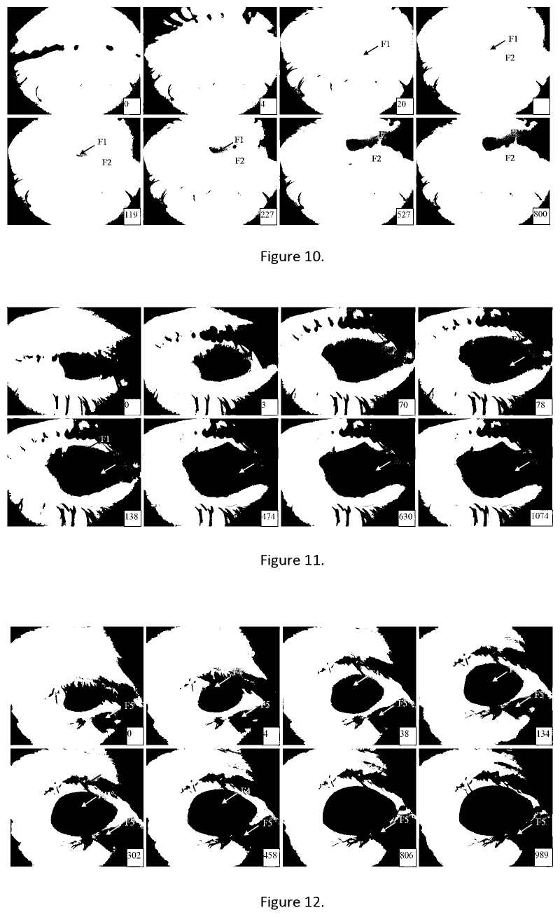

A subject with a dry eye condition was asked to sustain an eye opening for a period of time after an unforced blink. A thermographic video of the left eye of the subject was captured. As illustrated in FIG. 11, a detected tear film was formed immediately after the eye opening, but the detected shape, detected size, and detected position of the tear film began to change at around 0.7 seconds after the eye opening. This represents a different form of dry eye from that showing the characteristics seen in FIG. 10, and is less stable in the detected physical behaviour of the tear film than that of a normal eye (FIG. 1.)

### Example 11: Dry Eye Case Study 3

A subject with yet another dry eye condition was asked to sustain an eye opening for a period of time after an unforced blink. A thermographic video of the left eye of the subject was captured. As illustrated in FIG. 12, almost no detected tear film was formed immediately after the eye opening. The eye ball was exposed to the environment without being covered by a functional aqueous retaining tear film, evidenced by the gradually darkening grey scale on the ocular surface due to evaporative cooling.

### Example 12: Sjögren's Disease

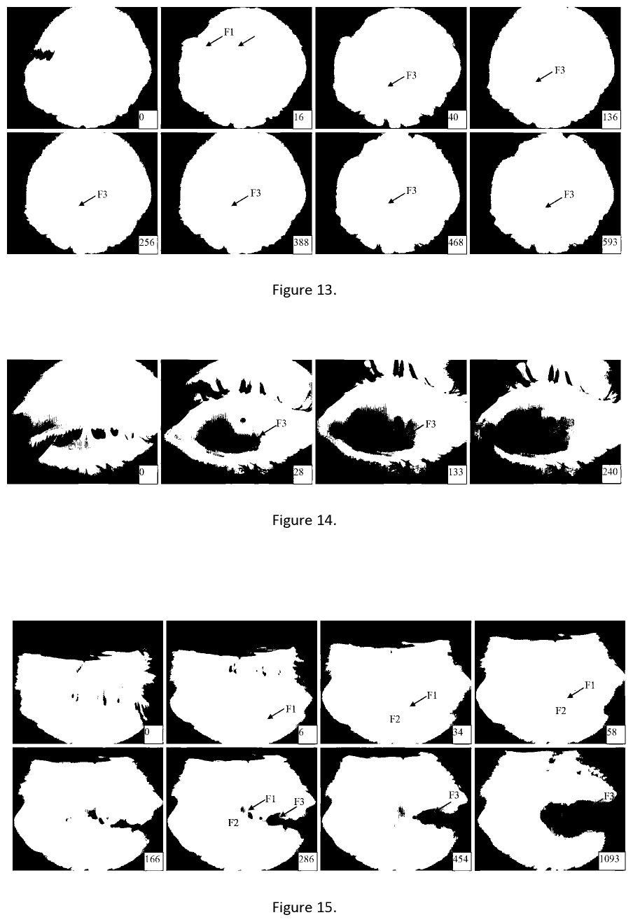

A subject with Sjögren's disease was asked to sustain an eye opening for a period of time after an unforced blink. A thermographic video of the right eye of the subject was captured. As illustrated in FIG. 13, a detected tear film was formed immediately after the eye opening, but the detected shape, detected size, and detected position of the tear film began to change at around 0.4 seconds around the bottom portion of the ocular surface after the eye opening. This change continued to grow toward the upper portion of the ocular surface.

### Example 13: Shingles

A subject with a shingles infection in the eye was asked to sustain an eye opening for a period of time after an unforced blink. A thermographic video of the right eye of the subject was captured. As illustrated in FIG. 14, a detected tear film was formed immediately after the eye opening. However, unlike detected tear film formed after an eye opening in a normal eye, the detected shape, detected size, and detected position of the tear film in the eye with a shingle infection has detected irregularity around the location of the shingles infection immediately after the eye opening. This detected irregularity expanded outward continuously.

### Example 14: Corneal Lesion

A subject with a corneal lesion was asked to sustain an eye opening for a period of time after an unforced blink. A thermographic video of the left eye of the subject was captured. As illustrated in FIG. 15, a detected tear film was formed immediately after the eye opening. However, unlike detected tear film formed after an eye opening in a normal eye, the detected shape, detected size, and detected position of the tear film in the eye with a corneal lesion began to illustrate detected irregularity around the location of the lesion at around 0.5 seconds after the eye opening. This detected irregularity began to settle down around that location after around 3 seconds.

### Example 15: Keratoconus

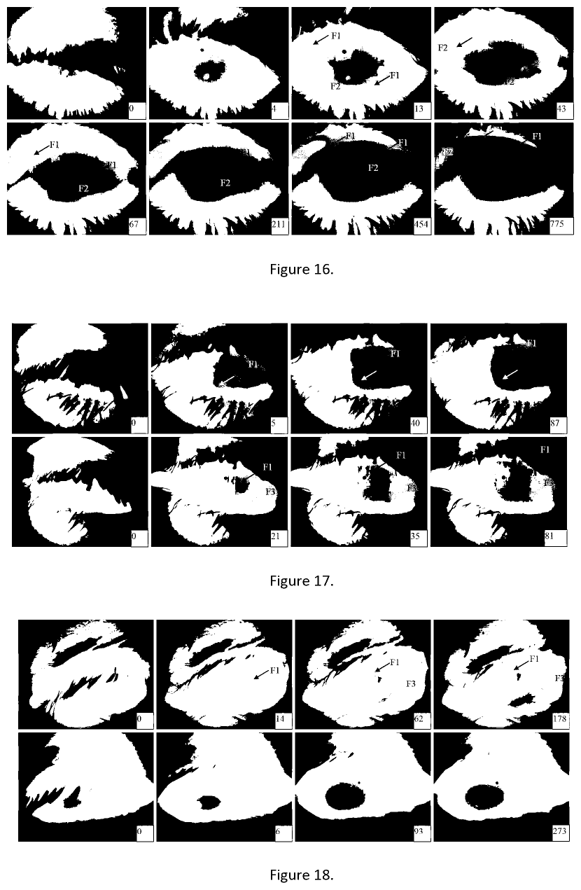

A subject with Keratoconus was asked to sustain an eye opening for a period of time after an unforced blink. A thermographic video of the left eye of the subject was captured. As illustrated in FIG. 16, a detected tear film was formed immediately after the eye opening. However, unlike detected tear film formed after an eye opening in a normal eye (FIG. 1), the detected shape, detected size, and detected position of the tear film in the eye illustrated irregularity during the formation of the tear film. This detected irregularity began to move upward continuously. After around 5 seconds, the detected irregularity spread across almost the entire ocular surface.

### Example 16: Specific Eye Drop 1

A subject with a dry eye condition was monitored before and after receiving a specific eye drop that had a lipid as the active ingredient. The subject was asked to sustain an eye opening for a period of time after an unforced blink. Two thermographic videos of the left eye of the subject with or without receiving the eye drop were captured. As illustrated in FIG. 17, before application of the eye drop (top row) a detected tear film was formed immediately after the eye opening, but the detected shape, detected size, and detected position of the tear film began to change at around 0.4 seconds after the eye opening. After application of the eye drop (bottom row) a tear film was formed immediately after the eye opening and the detected shape, detected size, and detected position of the tear film was similar to a normal tear film (FIG. 1).

### Example 17: Specific Eye Drop 2

A subject with a dry eye condition was monitored before and after receiving a specific eye drop that had a thickening polymer (carboxymethylcellulose) as the active ingredient. The subject was asked to sustain an eye opening for a period of time after an unforced blink. Two thermographic videos of the left eye of the subject with or without receiving the eye drop were captured. As illustrated in FIG. 18, before application of the eye drop (top row) a detected tear film was formed immediately after the eye opening, but the detected shape, detected size, and detected position of the tear film began to change at around 0.6 seconds after the eye opening. After application of the eye drop (bottom row) a film over the eye was present upon eye opening and the detected shape, detected size, and detected position of the film did not change over an extended time.

### Example 18: Blinking Exercise

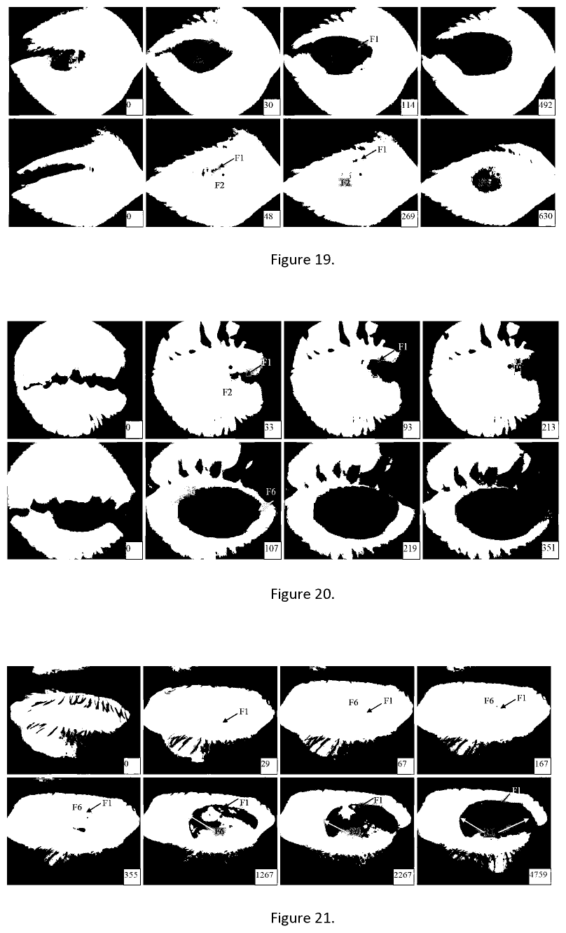

A subject with dry eye was asked to sustain an eye opening for a period of time after an unforced blink, before and after a blinking exercise regime for one week. Two thermographic videos of the right eye of the subject were captured. As illustrated in the upper and lower rows in FIG. 19, the detected shape, detected size and detected position of the tear film after the blinking exercise resemble that of a tear film of a normal eye, which is much more stable than the detected shape, detected size and detected position of the tear film before the blinking exercise regime.

It is known that during the eyelid closing phase of a blink cycle some aqueous is removed and it is replenished during eyelid opening (Sorbara et al 2004 Contact Lens Ant Eye 27:15-20; Khanal and Millar 2010 Nanomedicine. 6:707-713). For a normal natural tear film, at the beginning of the eye-opening phase of a blink, a darker narrow front across the ocular surface (Feature F1; in FIGS. 1, 10, 11, 13, 15-19), which moves from the lower eyelid towards the upper eyelid, can be seen. Being darker, this front appears to emit less thermal radiation and has lower emissivity than the surface of the eye it is spreading over. This may be in part related to the emissivity changes due to the lipid layer forming on the top of the tear film and in part related to the aqueous component of the tear film evaporating.

Following behind this dark front is a light grey region (Feature F2 in FIGS. 1, 10, 15, 16, 19) representing a newly formed complete tear film covering the ocular surface. Research has identified that the dark front is indicative of an aqueous component of the tear film that has not fully integrated in the tear film during its spread after opening the eye. It also represents changes in emissivity of the tear film due to the lipid front moving simultaneously. Since this part of the aqueous of the tear film is not fully integrated into the tear film it can evaporate, hence the small difference in thermal radiation compared to the surface of the eye the film has already spread over. Again, research has shown that in healthy subjects not affected by dry eye or other problems to the tear film, this front (Feature F1) moves up over the ocular surface and then disappears at the upper eye margin (FIG. 1).

The spreading front of the tear film (Feature F1) can be fast moving (within a tenth of a second from opening the eye until it reaches the top of the eye) or relatively slow (the same process over a second or more). These different velocities are due to differences of interaction of the aqueous component and lipid component of the tear film with the mucinous component of the tear film. Following behind this front in a normal healthy eye is the complete tear film (Feature F2 in FIG. 1). Also in healthy subjects, the resulting complete film does not show a substantial drop in thermal radiation or fluctuations in emissivity over time and over the entire air exposed surface of the eye (not becoming darker while the eye is open indicating a stable non-evaporative tear film; FIG. 1). In some subjects with abnormal tear films, the front of low thermal radiation does not reach the top of the eye, resulting in an area with low thermal radiation in those parts of the eye that the front has not moved over (FIG. 10, the low thermal radiation is attributed to enhanced evaporation in these areas).

In subjects with abnormal tear films, differences in thermal radiation can be seen in the region following the front (Feature F2, the light grey region between the front and the lower lid margin in the healthy tear film). In particular, dark zones (Feature F3 in FIGS. 11, 13-15, 17-19) appearing over time on the surface of the eye represent areas of high evaporation and therefore indicative of regions where the tear film covering the eye's surface is unstable. These darkened zones (Feature F3 in FIG. 11) can appear in cases where the front has moved to the top of the eye, and in cases where the dark front does not reach the top of the eye (FIG. 19, top row).

In the eyes of some subjects with an abnormal tear film, no zone can be seen moving over the eye, but instead there is a rapid decrease in thermal radiation of the ocular surface due to excessive evaporation (FIG. 12). In these cases no aqueous retaining tear film has formed. Extensive research has shown that for the spreading front (Feature F1) to be seen, the aqueous part of the tear film together with the tear film lipids released from the Meibomian glands have to interact with mucinous components of the tear film. A tear film devoid of the front (Feature F1) as seen in FIG. 12 as an example is indicative of an abnormal interaction in this respect. Another example of this is illustrated in FIG. 18 after an eye drop designed as a viscous tear substitute was applied to the eye.

The interaction of the aqueous and lipid components with mucin components of the tear film indeed slows down the tear film spreading over the eye to the extent that it becomes visible with the method disclosed in this invention. Thus, in a diseased state of the eye as seen in FIG. 12, the spread could be too fast to be registered. It is also possible that the overall differential in emissivity with regard to the lipid layer and in thermal radiation with regard to evaporation is too low to be registered (detected). An aqueous layer still covers the eye's surface directly after the blink as seen in the reflection of the image of the infrared camera (Feature F4 in FIG. 12) in the thermographs.

In cases as shown here in FIG. 12, where the tear film shows excessive evaporation, this image disappears indicating that the ocular surface has become dry (FIG. 12 at time 806). In the case of this subject the Meibomian glands were analysed to be functional and releasing adequate tear film lipids. There appears to be adequate aqueous in this case because aqueous can be seen spilling onto the outside of the lower eye lid (Feature F5).

Some embodiments of the invention use changes in detected physical behaviour of a tear film in a patient's eye, such as changes in the detected shape of the tear film, to make diagnoses or to develop or monitor treatment regimes for ocular disorders. The detected shape of the tear film in a normal eye, as shown in FIG. 1, immediately after an unforced blink, usually closely resembles the shape of the eye and this detected shape is relatively stable for the first few seconds after a blink. The detected shape of the tear film in an eye affected by dry eye disease, on the other hand, might be similar to the detected shape of the tear film in a normal eye but that shape changes either immediately or over time.

As shown in FIG. 10 and FIG. 11, in eyes affected by dry eye disease, the detected irregular shape along the moving front of the tear film, moves toward the central lateral line of the eye in the early part of the first second after a blink. As shown in FIG. 15, in an eye with a corneal lesion, an irregular shape is detected around the position of the corneal lesion immediately after a blink (potentially caused by the corneal lesion), and the detected shape of the tear film becomes more regular toward the end of the first second after a blink (potentially due to the spreading of the tear film).

As shown in FIG. 13, in an eye affected by Sjögren's syndrome, the detected shape of the bottom part of the tear film was regular along the edge of the ocular surface immediately after a blink then becomes irregular after around 1 second after a blink. As shown in FIG. 14, in a tear film of an eye after a shingles infection, the detected shape of the tear film is irregular at the place of infection immediately after a blink. As shown in FIG. 19, in a tear film of an eye after a blink following a week of eyelid blinking exercises, the detected shape of the tear film is more stable after the blink than before the eyelid blinking exercise.

As shown in FIG. 16, in an eye with keratoconus, the detected bottom shape of the tear film begins to show irregularity at around 0.4 second after a blink and this detected irregularity moves up and makes the detected shape of the whole tear film irregular at around 7.8 seconds after a blink. As shown in FIG. 17 after instilling an eye drop the detected shape of the tear film changes from that seen before instilling the eye drop and its detected shape and changes to its detected shape closely resemble those of the tear film of a normal eye (FIG. 1). As shown in FIG. 18 after instilling a different type of eye drop no shape of the tear film can be detected at any time. A similar lack of detected shape of a tear film was perceived in a highly evaporative dry eye condition as seen in FIG. 12.

Some embodiments of the invention use changes in detected physical behaviour of a tear film in a patient's eye such as the detected size of the tear film. The tear film covers the area of the eye's surface, which is uniform in emissivity and no changes to thermal radiation are detected when compared to a detected tear film of a subject with a normal tear film (Feature F2). It is observed that in general, as shown in FIG. 1, the detected size of a normal tear film does not change shortly after the eye is fully open. By contrast, eyes affected by some types of dry eye disease, have a detected tear film size that changes (FIGS. 10 and 11) while in other types of dry eye, there is no detected tear film area (FIG. 12). As shown in FIG. 15, in an eye with a corneal lesion, the detected size of the tear film is considerably smaller immediately after a blink. As shown in FIG. 19, in an eye after a week of eyelid blinking exercises, the detected size of the tear film remains stable for a longer period of time than before the eyelid blinking exercises. The application of tear drops in dry eye conditions (FIGS. 17 and 18) is able to restore the detected size of the tear film to that detected in a normal tear film (FIG. 1).

Some embodiments of the invention use changes in physical behaviour of a tear film in a patient's eye such as the detected position of the tear film. In a normal eye, as shown in FIG. 1, the detected tear film is spread across the ocular surface exposed to the air. As shown in FIG. 15, in an eye with a corneal lesion, the detected position of the tear film is away from the location of the corneal lesion. As shown in FIG. 14, in an eye with a shingles infection, the detected position of the tear film is away from the location of the shingles infection. As shown in FIG. 13, in an eye affected by Sjögren's syndrome, the detected position of the tear film is away from the bottom part of the eye over the first second immediately after a blink. As shown in FIG. 16 the detected position of the tear film is away from the protruding cornea in keratoconus. As shown in FIGS. 17 and 18, the application of eye drops can detected repositioning of the tear film.

As explained above, the detection of physical behaviour is, in preferred and alternative embodiments, achieved by visualising or observing captured data sets from a patient's eye. In some such embodiments, the visualisation or observation can be done on a screen, in recorded digital or analogue form, or in printed form, for example, in pictures and/or diagrams, all of these mechanisms being adopted with or without magnification means adapted to magnify the captured data set.

In some embodiments, the detection of physical behaviour occurs through capturing emissions and/or remissions within wavelengths from throughout the and/or the electromagnetic radiation spectrum. In some preferred embodiments, detection occurs through infra-red emissions and/or remissions and visible light emissions and/or remissions.

In some embodiments, detected tear film behaviour is conducted once off, continuously, and/or periodically. As is explained in more detail in this patent specification, in preferred and alternative embodiments, the capturing of the tear film behaviour is accomplished by observation, monitoring or recording. Preferably, practitioners can interrogate the observed detected tear film behaviour together with other comparative tear film behaviour to diagnose, develop or monitor a treatment regime for and ocular condition.

Captured tear film physical behaviour shown in the figures is recorded by practitioners using computer software. The form of the recording can be video and/or pictures stored in digital and/or analogue form or pictures and/or diagrams in printed form. The practitioners then use the recorded tear film physical behaviour together with other comparative tear film behaviour to diagnose, develop or monitor a treatment regime for ocular condition.

The capturing of the tear film physical behaviour shown in the figures began shortly after a blink, before the eye was fully open after a blink, because a substantial part of tear film physical behaviour occurs shortly after a blink, the time during which a tear film is formed.

As shown in the figures, the time period of capturing of relevant tear film physical behaviour could vary. As shown in FIG. 10, FIG. 11 and FIG. 12, the capturing of tear film physical behaviour in an eye with dry eye condition, may last from 8 to 11 seconds following commencement of the capturing. As shown in FIG. 13, the capturing of tear film physical behaviour in an eye with Sjögren's syndrome, may last from 1.3 to 6 seconds following commencement of the capturing. As shown in FIG. 14, the capturing of tear film physical behaviour in an eye after a shingles infection, may last from 0.3 to 3 seconds following commencement of the capturing. As shown in FIG. 15, the capturing of tear film physical behaviour in an eye having a corneal lesion, may last from 0.3 to 11 seconds following commencement of the capturing. As shown in FIG. 16, the capturing of tear film physical behaviour in an eye with keratoconus, may last from 0.4 to 8 seconds following commencement of the capturing.

As shown in FIG. 17 and FIG. 18, the capturing of tear film physical behaviour in an eye before and after application of a specific eye drop, may last from 0.3 to 3 seconds following commencement of the capturing. As shown in FIG. 19, the capturing of tear film physical behaviour in an eye before and after performing eyelid blink exercises, may last from 0.3 to 7 seconds following commencement of the capturing.

The capturing shown in the figures can be conducted at different time with different settings, in which multiple segments of tear film physical behaviour are captured.

Depending on the type of ocular condition and/or the type of diagnosis, development or monitoring of eye condition, further capturing of tear film physical behaviour may occur again between 6 months and about one year following the preceding capturing time. In some other embodiments, the second or further capturing may commence periodically following the preceding capturing time, wherein the period may be a day, a week, a month, a quarter, a year, or more. Alternatively, the second or further capturing time is decided by the practitioners or the subjects depending on various considerations such as efficiency, diagnostic accuracy, response to or reaching a certain stage of treatments, exercise regime, symptoms, feeling, availability, and developing problems.

The captured tear film physical behaviour shown in the figures can also be used as one or multiple set/s of comparative data needed to diagnose, develop or monitor eye condition, along with the captured tear film physical behaviour. The captured tear film physical behaviour shown in the figures could be taken from the same or different subjects, at a different time or under a different condition or a combination of them.

If the captured tear film physical behaviours shown in the figures are used to diagnose, develop or monitor eye condition, one or more comparative data set/s may be identified as knowledge base of practitioners or people performing this invention. This knowledge base includes training, studying, or experience of such person. The knowledge may be in the form of the memory of the person, or data in printed or digital form such as texts, tables, diagrams, pictures, imaging or videos in relation to tear film physical behaviour that assist the person to diagnose, develop or monitor an eye condition. The captured tear film physical behaviour shown in the figures can themselves by used as the one or more comparative data set/s.

The analysis of the captured tear film physical behaviour and the comparative data set/s can be carried out by examining the diagnostic characteristics illustrated. Identifying similar or same tear film physical behaviour such as detected size, detected shape, and detected position would indicate closely related diagnostic characteristics.

In some preferred embodiments, a suitable infrared sensitive camera includes:
a. a detector for wavelengths in the bands from 2 μm to 14 μm;
b. a frame rate above 10 frames per second;
c. a spatial resolution detector of at least 320×240 pixels;
d. a pitch resolution of 17 μm or less;
e. a thermal sensitivity of at least 35 mK.

The material of the lens system of the camera could be gallium, zinc selenide or zinc sulfide. The lens material should be a material with high thermal transmittance and for practical reasons should not be affected by ambient humidity or temperature.

The photodetector of the camera can be cooled and of different materials which include, but are not limited to, indium antimonide, indium arsenide, mercury cadmium telluride, lead sulfide, lead selenide. A common cooling mechanism that would be used is a Stirling engine cryocooler, but others such as gas coolers could also be used. The photodetectors include high band gap semiconductors such as quantum well infrared photodetectors. The digital information from the camera is processed by appropriate software.

If desired, software could be used to enhance the captured tear film physical behaviour shown in the figures. For non-limiting examples, software computerising a mean of multiple frames into one frame could be used to increase the temperature sensitivity (reduce the noise); comparing neighbouring pixels and performing statistical analysis could be used for contrast enhancement or other enhancements. This software can be installed in a computer, in a camera or a standalone device.

a typical eye examination session proceeds as follows: The camera system is started and if necessary the camera is cooled to operational requirements. A computer with relevant software is started.

The patient is seated in front of the camera and asked to place his/her chin in a chin rest. The chin rest is adjusted for the patient to sit comfortably. The camera is adjusted to place it horizontally in front of the eye of the patient.

The camera can either work with a fixed focus or an adjustable focus. In the case of a fixed focus, the camera is moved on the horizontal axis to or away from the eye of the patient to get the thermographic picture in focus. If the focus of the camera is not fixed, additional adjustments can be made using the focal lens of the camera.

After the eye is in focus of the camera, the operator of the tear film thermographer gives instructions to the patient for blinking regimes and physical behaviour of tear film is captured and recorded as desired. This process can be repeated multiple times if needed or preferred.

The captured tear film behaviour is then analysed against a comparative tear film behaviour recorded in a database or a printed manual. A diagnosis is then made, or a treatment regime is developed or monitored, for the subject.

The camera system can be fully motorized, manual, semi-autonomous or autonomous in operation. Such a tear film thermographer could be a stand-alone system or attached to another ophthalmic instrument allowing for movement of the camera into the right position in front of the eye of a patient. Such a system could be a slit lamp to which an infrared sensitive camera is attached so it can be moved as required.

C. Use of the Method in Contact Lens Selection

There is a wide range of different contact lenses available and these differ in size, thickness, shape, material, surface characteristics, other material characteristics and intended purpose (reusable, extended wear, dailies, weeklies, monthlies, and cosmetic wear).

The inventors undertook a series of experiments reflecting how the method of the present invention can be used to improve currently adopted processes for contact lens selection. The tear film behaviour with and without an instilled contact lens was recorded using a thermographic camera with an 640×512 indium antimonide detector array with a pixel pitch of 15 μm, a temperature resolution of 20 mK, a 50 mm lens with a 20 mm extension ring run with windowing at 100 Hz. The experiments were carried out in a controlled environment at a temperature of 23° C. and humidity of 45%. The thermographs in FIGS. 20-24 illustrating findings from Examples 19-23 are grey scaled, where darker grey represents less thermal radiation.

### Example 19: Contact Lens Case Study 1

A normal subject was asked to sustain an eye opening for a period of time after an unforced blink. Afterwards the subject inserted an extended wear contact lens. A thermographic video of the eye of the subject was captured. As illustrated in FIG. 20, a detected tear film was formed immediately after the eye opening, but the detected shape, detected size, and detected position of the tear film was normal (FIG. 1) without a contact lens (top row). Without the contact lens, the dark band (F1) with a light grey sheet (F2) following behind moves relatively rapidly and completely up the ocular surface, and afterwards there is virtually no change in the surface for many seconds if the eye is kept open. Shortly after placing an extended wear contact lens in the eye (bottom row) the detected shape, detected size and detected position of the tear film are altered. The dark band moving up the eye and its associated grey sheet are not detected. Instead, the region of the contact lens can be seen (margins indicated by F6) which slowly darkens with time, indicating that the tear film was not formed correctly in this region resulting in excess evaporative cooling.

### Example 20: Contact Lens Case Study 2

A normal subject inserted a daily wear contact lens. A thermographic video of the eye of the subject was captured. As illustrated in FIG. 21, shortly after placing a daily wear contact lens in the eye, the detected shape, detected size, and detected position of the tear film are altered compared with a normal tear film (FIG. 1). An uneven dark zone (F1) moves very slowly upwards and a grey non-uniform shape rises behind this irregular margin to form a grey cloud over the contact lens. The right margin/s of the contact lens is/are indicated (F6). The region of the contact lens not covered by the grey cloud becomes darker, indicating evaporative cooling from the surface in this region of the contact lens.

### Example 21: Contact Lens Case Study 3

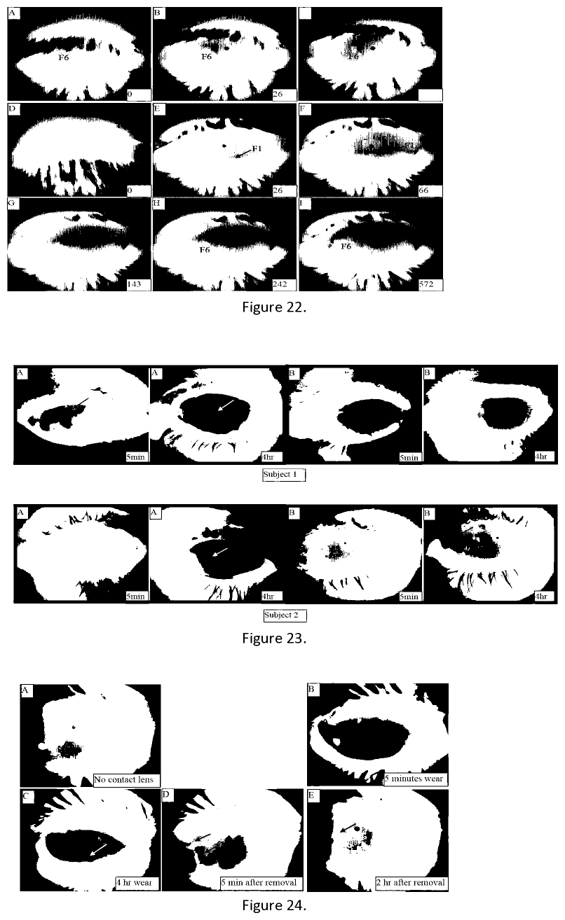

A normal subject inserted a daily disposable contact lens of the same brand into each eye (FIG. 22). A thermographic video of the eye of the subject was captured. As illustrated in FIG. 22, after 6 hours of wear the detected shape, detected size and detected position of the tear film is altered in one eye (FIG. 22D-I), while in the other eye (FIG. 22, A-C) the detected shape, detected size and detected position of the tear film is not altered compared to a normal tear film (FIG. 1). In the case of the altered tear film, an uneven dark zone (F1) moves very slowly upwards and a grey non-uniform shape rises behind this irregular margin to form a grey cloud over the contact lens. The area of the margin (F1) not covered by the grey zone progressively gets darker with time indicating evaporative cooling from this region covered by the contact lens. The subject observed discomfort in this eye. A margin of the contact lens is indicated (F6).

### Example 22: Contact Lens Case Study 4

The effects on the tear film of a weekly contact lens (A) and a monthly contact lens (B) on the tear film of two subjects (top row Subject 1 and bottom row Subject 2) just after insertion (approximately 5 minutes after insertion) and then after 4 hours of wear were monitored. Thermographic videos of the eye of the subjects were captured during each period for each subject (FIG. 23). All pictures were taken approximately 2 seconds after eye opening. The weekly contact lens (A) has an immediate effect on the detected shape, detected size and detected position of the tear film of Subject 1 (arrows) but no effect on the detected shape, detected size or detected position of the tear film of Subject 2 compared with a normal tear film (FIG. 1). However, after 4 hours of wear, the tear film of both wearers has progressively changed detected shape, detected size and detected position of the tear film compared with a normal tear film (FIG. 1). The weekly contact lenses in both Subject 1 and Subject 2 were tolerable after 4 hours. The monthly contact lens (B) affects the detected shape, detected size and detected position of the tear film of Subject 1 initially, but resembles the tear film of a normal tear film after 4 hours wear. For Subject 2 the monthly contact lens (B) has little effect on the detected shape, detected size and detected position of the tear film compared with a normal tear film (FIG. 1). However, after 4 hours of wear, a distinct patch in the superior medial region of the eye is not being covered properly by the tear film (arrow). The Subject reported discomfort in this area.

### Example 23: Contact Lens Case Study 5

The effects on the tear film of an extended wear contact lens was monitored before, during, and after removal of the contact lens. Thermographic videos of the eye of the subject was captured during each period (FIG. 24). All pictures were taken approximately 2 seconds after eye opening. Before insertion of the contact lens, the detected shape, detected size and detected position of the tear film was normal (A). Shortly after inserting the contact lens the detected shape, detected size and detected position of the tear film is severely disrupted as indicated by the dark central region (B). After 4 hours of wear, the area of the tear film grows (C margin indicated by arrow). Shortly after removal of the contact lens there is some disruption to the tear film (D arrows). Two hours after removing the contact lens, the tear film has returned to normal (E). Note that the detected shape arrowed in E is a thermal reflection of the clinician.

It is estimated that about half of contact lens wearers experience ocular discomfort due to wearing contact lenses. This often extends to contact lens wearers giving up wearing contact lenses. Despite this condition impacting millions of contact lens wearers worldwide, there is a paucity of consensus and standardization in the scientific and clinical communities on the characterisation of contact lens discomfort and the effects wear on the tear film and the eye surface (Nichols et al. 2013. IOVS TFOS 7-13).

The method utilised in the examples above provide, in preferred embodiments, a mechanism for evaluating the effect of contact lens wear on the detected shape, detected size and detected position of the tear film and relating this to comfort and discomfort by the wearer.

As the examples show, the effect of different contact lenses on the detected shape, detected size and detected position of the tear film. It is also evident that the effect of the contact lens on the detected shape, detected size and detected position of the tear film can vary between individuals and over time. In some subjects, using specific contact lens types, at particular times after insertion, the detected shape, detected size and detected position of the tear film had a normal appearance.

These experimental findings are consistent with the fact that different contact lenses have different coatings or surface treatments affecting the wettability and binding of tear film components to the contact lens. Different subjects have different tear film composition and elements from the tear film can interact and adhere to contact lens surfaces during wear and so change the characteristics of the contact lens surface and hence contact lenses vary in their interaction with the tear film.

In preferred embodiments, an iterative process is used to determine a brand and style of contact lens that least interferes with the normally detected shape, detected size and detected position of a tear film in a subject.

Preferably, contact lenses that least interfere with the detected normal shape, detected normal size and detected normal position of the tear film are the most comfortable for the wearer. Accordingly, in some embodiments, the method of the present invention provides a mechanism for selecting a suitable contact lens for a subject. In other embodiments, the method of the present invention provides a mechanism for evaluating the effects of wearing a contact lens on the detected shape, detected size and detected position of the tear film, including after the contact lens had been removed. Preferably, these mechanisms enable a determination of the preferable wearing periods of contact lenses and rest periods from contact lens wear for subjects.

Moreover, in preferred and alternative embodiments, it can be apparent that:
a. in the example shown in FIG. 23, different contact lenses can lead to different detected physical behaviours in the tear film of the same subject,
b. in the example shown in FIG. 22, the same contact lens can lead to different detected physical behaviours in the tear films of each of the two eyes in the same subject,
c. in the example shown in FIG. 23, the same contact lens in the same eye of a subject can lead to different detected physical behaviours in the tear film after time of being instilled,
d. in the examples shown in FIGS. 20 to 24, the same type of contact lens can lead to different detected physical behaviours in the tear films of different subjects, and
e. in the example shown in FIG. 24, the detected physical behaviours in the tear film of a subject appear different after a contact lens has been removed.

As explained, in currently used processes of recommending and choosing the correct contact lens for a patient, a clinician typically initially determines the desired purpose for contact lens, the patient hygiene, the ability of the patient to insert and remove a contact lens, and the power and shape of the contact lens required. This process narrows the number of brands suitable for a given patient and then trial lenses of those brands are fitted to the patient. The final selection is made via an iterative process predominantly based on patients' perception of comfort.

Preferred and alternative embodiments of the present invention provide an objective means for determining a suitable contact lens, following the initial narrowing of choices.

In some embodiments, the clinician analyses the detected physical behaviour of tear film in the eye/s of the patient prior to fitting a trial lens of the same brand and type into each eye. After initial tearing has subsided (within about 5 minutes from instillation of the trial contact lens/es), the clinician can compare the detected physical behaviour of the tear film with the detected physical behaviour of the tear film before such fitting, and/or with other comparative data set/s.

In some preferred embodiments, the patient wears the contact lens/es for a predetermined or preferred period, following which re-examination/s at different time period/s after fitting the contact lenses are conducted. Such time periods could be 2 hours, 4 hours, 6 hours, 8 hours, 24 hours or longer.

Some such embodiments provide that during re-examination, the detected physical behaviour of the tear film following instillation of the contact lens/es is again compared with the detected physical behaviour of the tear film before fitting, with other data set/s captured of the physical behaviour of the tear film after fitting the or other contact lens/es, and/or with other comparative data set/s. Preferably, the patient is asked about their relative comfort at each recording time point.

Whether following a first examination or each or further examination, the contact lens/es are removed and the detected physical behaviour of the tear film recorded (preferably about 5 minutes after). The detected physical behaviour of the tear film is then preferably compared with the detected physical behaviour of the tear film before fitting, with comparative data set/s recorded after fitting the contact lens, and/or with other comparative data set/s.

A particularly preferred contact lens for selection is one the removal of which allows the detected physical behaviour of a tear film to show essentially immediate re-establishment of a normal tear film. In embodiments wherein the detected physical behaviour of the tear film is disrupted after removal of the contact lens, then a lesser preferred contact lens for selection is one the removal of which allows the detected physical behaviour of the tear film to be close to undisrupted or only mildly disrupted, with re-establishment of normalcy in a relatively short period of time (for example in less than 2 minutes).

For some such embodiments, typical intervals for measurement after removal of the contact lens are from about 10 minute intervals up to about hourly intervals. Persons skilled in the art will appreciate that preferred time intervals for measurement after removal of the contact lens may vary from eye to eye, contact lens to contact lens, and from patient to patient.

According to some embodiments, a preferred contact lens for selection is one which resulted in a detected physical behaviour of a normal tear film initially on instillation of the contact lens and then continuing over time or, in some embodiments, does not result in a change in the detected physical behaviour of the tear film from before the fitting the contact lens/es.

In some embodiments, a less preferred contact lens for selection is one which does not allow the detected physical behaviour of the tear film to show normal formation of a normal tear film initially following instillation, but allows the detected physical behaviour of the tear film to show either a complete or partial tear film formation over time.

In further embodiments, a still less preferred contact lens for selection is one which results in the detected physical behaviour of a tear film appearing normal initially and then deteriorating over time. Preferred and alternative embodiments disclose that the relatively quicker deterioration occurs, the less preferred a contact lens will be for selection.

In yet still further embodiments, a least preferred contact lens for selection is one for which the detected physical behaviour of tear film appears disrupted initially and remains disrupted over time.

In some preferred and alternative embodiments, the method steps outlined are repeated with different contact lenses to establish the brand of contact lens that achieves selection of a preferred contact lens. In some embodiments, a different brand of contact lens is preferable in each eye.

In addition, in preferred embodiments, long term effects (for example, months to years) of contact lens wear on detected physical behaviour of the tear film are also monitored and/or considered. In some such embodiments, this can be achieved by comparing data set/s of the tear film created during the initial fitting and selection process.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

It is to be noted that, throughout the description and claims of this specification, the word “comprise” and variations of the word, such as “comprising” and “comprises”, is not intended to exclude other variants or additional components, integers or steps. Modifications and improvements to the invention will be readily apparent to those skilled in the art. Such modifications and improvements are intended to be within the scope of this invention.

1. A method of diagnosing, or developing or monitoring a treatment regime for, an ocular condition in a subject based on detected physical behaviour in a tear film, or lack of tear film, in the subject's eye, the method comprising the steps of:
a. capturing from the subject's eye at least a first captured data set;
b. analysing the at least a first captured data set and thereby detecting physical behaviour in the tear film; and
c. diagnosing, or developing or monitoring a treatment regime for, the ocular condition based on the detected physical behaviour of the tear film.

2. A method of diagnosing, or developing or monitoring a treatment regime for, an ocular condition in a subject based on detected physical behaviour in a tear film, or lack of tear film, in the subject's eye, the method comprising the steps of:
a. capturing from the subject's eye at least a first captured data set;
b. identifying at least a first comparative data set;
c. analysing the at least a first captured data set relative to the at least a first comparative data set, thereby detecting physical behaviour in the tear film; and
d. diagnosing, or developing or monitoring a treatment regime for, the ocular condition based on the detected physical behaviour of the tear film.

3. The method of claim 1 wherein the detected physical behaviour is defined by one or more characteristics of the tear film, or lack of tear film, selected from the group consisting of: shape, size, and position.

4. The method of claim 3 wherein detection of the physical behaviour is achieved at a macroscopic level.

5. The method of claim 1 wherein the step of capturing is achieved by one or more mode selected from the group consisting of: observing, monitoring, and/or recording.

6. The method of claim 1 wherein the first captured data set includes data reflecting detected physical behaviour of a tear film, or lack of tear film, in the subject's eye identified at a first predetermined time and/or over a predetermined period of time or a plurality of predetermined times.

7. The method of claim 6 wherein the first predetermined time for identifying a detected physical behaviour is between about 1×10⁻² and about 2×10⁻¹ seconds following commencement of the step of capturing.

8. The method of claim 1 wherein the capturing step further includes capturing at least a second captured data set.

9. The method of claim 8 wherein the second captured data set includes data reflecting detected physical behaviour of a tear film, or lack of tear film, in the subject's eye identified at a second predetermined time and/or at a further plurality of predetermined times.

10. The method of claim 9 wherein the second predetermined time for identifying a detected physical behaviour is between about 2×10⁻² and about 2×10⁻¹ seconds following the first predetermined time.

11. The method of claim 9 wherein the second predetermined time for identifying a detected physical behaviour is between about 6 months and about one year following the first predetermined time.

12. The method of claim 9 wherein the further plurality of predetermined times occurs about monthly, quarterly, or annually, after the second predetermined time.

13. The method of claim 6 wherein the first captured data set or one or more further captured data set/s include/s data reflecting detected physical behaviour of a tear film, or lack of tear film, in the subject's eye identified over a predetermined period of time, which period, following commencement of the step of capturing, is selected from the group consisting of: about 0.00 to about 1.00 second, about 0.00 to about 3.00 seconds, about 0.00 to about 6.00 seconds, about 0.00 to about 10.00 seconds, about 0.00 to about 15.00 seconds, about 0.00 to about 30.00 seconds, about 1.00 to about 7.00 seconds, about 3.00 to about 12.00 seconds, and about 6.00 to about 20.00 seconds.

14.-16. (canceled)

17. The method of claim 2 wherein the step of identifying is achieved by referencing from at least a predetermined knowledge base, and/or observing, monitoring, measuring and/or recording from a predetermined source.

18. The method of claim 17 wherein the predetermined knowledge base includes information based on training, study and/or experience of a person in relation to detected physical behaviour in a tear film, or lack of tear film, in a subject's eye, which person is undertaking the diagnosis of, or development or monitoring of a treatment regime for, an ocular condition in the subject.

19. The method of claim 17 wherein the predetermined source includes one or more data sets from the group consisting of:
a. any second of further captured data set, and/or
b. one or more data sets from the group consisting of photographs, video footage, medical or scientific imaging, and/or diagrams relating to detected physical behaviour in a tear film, or lack of tear film, in the subject's or other subjects' eyes.

20. The method of claim 1, wherein the step of analysing includes evaluating the at least a first captured data set relative to the at least a first comparative data set to identify at least a first set of diagnostic characteristics.

21. The method of claim 20 wherein the at least a first set of diagnostic characteristics is selected from the group consisting of: absence or presence of a possible ocular condition, differential diagnosis for a possible ocular condition, relative suitability of an ocular orthosis, relative efficacy of a treatment regime, and/or relative merit in maintaining, varying or ceasing a current treatment regime.

22.-29. (canceled)

30. A method of selecting a contact lens for a subject, the method comprising:
a. capturing from a first eye of the subject a first captured data set, the first captured data set including detected physical behaviour of the tear film in the first eye;
b. identifying a first test contact lens and having the first test contact lens instilled in the first eye;
c. after a predetermined or preferred first period of time, capturing from the first eye a second captured data set, the second captured data set including detected physical behaviour of the tear film of the first eye with the first test contact lens instilled;
d. analysing the second captured data set relative to the first captured data set and/or a comparative data set; and
e. evaluating the relative suitability of the first test contact lens to be selected as the contact lens for the subject.

31. The method of claim 30, further including, after a predetermined or preferred second period of time, capturing from the first eye a third captured data set, the third captured data set including detected physical behaviour of the tear film of the first eye after the first test contact lens has been removed following an instilled period.

32.-50. (canceled)
